SEQUENCE LISTING (22 sequences)

SEQ ID NO: 1 - DNA, 20 bp, Artificial Sequence
  Synthetic Nucleotide Sequence primer rtTA sense
gcaaaagaggaaagagagac

SEQ ID NO: 2 - DNA, 20 bp, Artificial Sequence
  Synthetic Nucleotide Sequence primer rtTA sense
ataagggaatggttgggaag

SEQ ID NO: 3 - DNA, 20 bp, Artificial Sequence
  Synthetic Nucleotide Sequence primer HA-hHuR sense
tttacccctacgacgtcccc

SEQ ID NO: 4 - DNA, 20 bp, Artificial Sequence
  Synthetic Nucleotide Sequence primer HA-hHuR antisense
tccttcgcggtcacgtagtt

SEQ ID NO: 5 - DNA, 20 bp, Artificial Sequence
  Synthetic Nucleotide Sequence primer mHuR/Elavl1 sense
atgtctaatggttatgaaga

SEQ ID NO: 6 - DNA, 20 bp, Artificial Sequence
  Synthetic Nucleotide Sequence primer mHuR/Elavl1 antisense
agctttgcagattcaacctc

SEQ ID NO: 7 - DNA, 17 bp, Artificial Sequence
  Synthetic Nucleotide Sequence primer mGAPDH sense
tcttcttgtgcagtgcc

SEQ ID NO: 8 - DNA, 19 bp, Artificial Sequence
  Synthetic Nucleotide Sequence primer mGAPDH antisense
actccacgacatactcagc

SEQ ID NO: 9 - DNA, 26 bp, Artificial Sequence
  Synthetic Nucleotide Sequence primer mTNF sense
cacgctcttctgtctactgaacttcg

SEQ ID NO: 10 - DNA, 26 bp, Artificial Sequence
  Synthetic Nucleotide Sequence primer mTNF antisense
ggctgggtagagaatggatgaacacc

SEQ ID NO: 11 - DNA, 20 bp, Artificial Sequence
  Synthetic Nucleotide Sequence primer mCOX2 sense
atgctcttccgagctgtgct

SEQ ID NO: 12 - DNA, 20 bp, Artificial Sequence
  Synthetic Nucleotide Sequence primer mCOX2 antisense
gcccacatctatgcttttca

SEQ ID NO: 13 - DNA, 20 bp, Artificial Sequence
  Synthetic Nucleotide Sequence primer mTTP sense
cagagcctccagtcgatgag

SEQ ID NO: 14 - DNA, 20 bp, Artificial Sequence
  Synthetic Nucleotide Sequence primer mTTP antisense
gaagatggggagacgccttg

SEQ ID NO: 15 - DNA, 25 bp, Artificial Sequence
  Synthetic Nucleotide Sequence primer mIL-6 sense
tggagtcacagaaggagtggctaag

SEQ ID NO: 16 - DNA, 25 bp, Artificial Sequence
  Synthetic Nucleotide Sequence primer mIL-6 antisense
tctgaccacagtgaggaatgtccac

SEQ ID NO: 17 - DNA, 20 bp, Artificial Sequence
  Synthetic Nucleotide Sequence primer mIL-1beta sense
ctgaagcagctatggcaact

SEQ ID NO: 18 - DNA, 20 bp, Artificial Sequence
  Synthetic Nucleotide Sequence primer mIL-1beta antisense
ggatgctctcatctggacag

SEQ ID NO: 19 - DNA, 18 bp, Artificial Sequence
  Synthetic Nucleotide Sequence primer mTGFbeta1 sense
ttcctgctcctcatggcc

SEQ ID NO: 20 - DNA, 18 bp, Artificial Sequence
  Synthetic Nucleotide Sequence primer mTGFbeta1 antisense
cttgcaggagcgcacgat

SEQ ID NO: 21 - DNA, 755 bp, Artificial Sequence
  Synthetic Nucleotide Sequence GenBank NM_013693, bases 865-1615
agggaatgggtgttcatccattctctacccagcccccactctgacccctttactctgacccctttattgtctactcctcagagcccccagtctgtgtccttctaacttagaaaggggattatggctcagagtccaactctgtgctcagagctttcaacaactactcagaaacacaagatgctgggacagtgacctggactgtgggcctctcatgcaccaccatcaaggactcaaatgggctttccgaattcactggagcctcgaatgtccattcctgagttctgcaaagggagagtggtcaggttgcctctgtctcagaatgaggctggataagatctcaggccttcctaccttcagacctttccagactcttccctgaggtgcaatgcacagccttcctcacagagccagcccccctctatttatatttgcacttattatttattatttatttattatttatttatttgcttatgaatgtatttatttggaaggccggggtgtcctggaggacccagtgtgggaagctgtcttcagacagacatgttttctgtgaaaacggagctgagctgtccccacctggcctctctaccttgttgcctcctcttttgcttatgtttaaaacaaaatatttatctaacccaattgtcttaataacgctgatttggtgaccaggctgtcgctacatcactgaacctctgctccccacgggagccgtgactgtaattgccctacagtcaattgagagaaataaa

SEQ ID NO: 22 - DNA, 2237 bp, Artificial Sequence
  Synthetic Nucleotide Sequence GenBank NM_011198, bases 1916-4116
aagtctactgaccatatttatttatttatgtgaagaatttaatttaattatttaatatttatactgaattttttttcatgtaacatcttccataacagaaggcaatgttcttgaacaatgttacatttgtgaagattccctccggtgtttgtcctttaaatatgtgttacctgaaactgaaaggaaatcagcattcattcctctacataagccagtgagaagggaaatgaattttgatatctttatacttgaatttcagatcatgattagcttaacaagaaccaaggaaaaatgtatgaatatgtgagtgttgttacaagatgaaaaatgctgcaggtatcaacactgttggttacaactgtgtcttctttactatgataggagcatgtaatgtggaattcttcttaaatcttgcatatctttatctcatcaaacaaaggggtccaagttcagttttaaataagcatttaaggcagatactgacaacaatctcattttttaaaatgttgtcttgagacaaataatttgaaatttctaaattgggagtttgaatcacttttgaaagctcttactttcttaagctgtcaggtttgtaccgacatggagtaaacagctatcataaacgtaaatctccaaaactagtagaaattatgtcatgattgatggttaagataccatgtcagggattgtcttttcttagaagtagtgaaagctacttactatgacaatcagaccttccttgtatgtcaaaatgctggtgtggaaggtggtggagcccgtgctgctctgtcttaactatgagtgtgagctttaaagctcgttgatgagtggtagccagcaaagcctagagcaacaaaagcttctacaaaggaactaaccaagaacaaagaagggttcccaattaaagatcacattcagggttaaacttccaaaggagacatcctgatcctggttttgtgctggcctggtactcagtaggtttttgctgtgaggttaaagacttgccaggctgaacttcgaaacagtttttctgttgcacagtatgatgtaacagtccatctctcaatgcaataggtatcagtggcctcgtgagcttcttcacaatattgatatgtcttccagcccattgaacctggactgcagaaggccccatgtcatgtgtgagctcagcctggatgccagcattgctgctcctcttagttccgtttctcgtggtcactttactacgagaaacgctgattgggttttcgtagctgtgttccaggtttttagtatcagaactattctttctttaacctctattcatattttctctacttgaagttttacattcaggaaaacctcagctcaggactactatgtacctcccctttggagggaaaaattattttaggtaaaaggcaaaaattttttaaaaatatttttatttataattatatggaagggccctaccaagatgctagaaatatagggagttcctgacaagaaatttccattcttattctgaagaattgctttcttacttaaaaacaaagacagtttgtgagtagttctgggcaatagggataaatataaaacaataatgatgatcattttctacatctcattatcagctgaggtactgtatattactgaatttattgaagatagttatgtcttttagacattgttgttataaactatgtttaagcctactacaagtgtttcttttttgcattatgttggaattgatgtaccttttttatgattacctctctgaactatggtgtgaacaatcaaacaaaatgatgagattaacgttcatggataaattctaagaaaactagtgtatttttttgaaaagtttgaagttagaacttaggctgttggaatttacgcataaagcagactgcatagatccaatattgactgacccaagcatgttataaagactgacattttagacattttgaaggccctgtaagtgtttattaattagttagaacttaattgattaaaaaatatatccaaagcactataggcattagaattcgggcatcaagaaatgatgacaaataatactgttatttatataaataactaaaaagggtgtctaatgaagaaatatattttattacaaagaaattataaaacattttgaagattatatgctttaaaagtttaagattgaaaaaataatcaaccttagaaaaaatgtataaaaatatataaattgttaatgtcattgattaaaaaaa

(19) United States
(12) Patent Application Publication
(10) Pub. No.: US 2008/0249047 A1
(43) Pub. Date: Oct. 9, 2008

(54) Modulation of Inflammation Through Modulation of Elavl1/HuR Expression

(76) Inventors: Dimitrios Kontogiannis, Peiraias (GR); Vassiliki Katsanou, Alimos (GR)

Correspondence Address: HOFFMANN & BARON, LLP, 6900 JERICHO TURNPIKE, SYOSSET, NY, 11791, US

(21) Appl. No.: 11/855,831

(22) Filed: Sep. 14, 2007

Related U.S. Application Data
(60) Provisional application No. 60/845,137, filed on Sep. 15, 2006.

(51) Int. Cl. A61K 48/00; A61P 29/00

(52) U.S. Cl. 514/44

(57) ABSTRACT

In the present invention modulation of expression of a member of the Elavl1/Hu family is used to modulate at least translation of specific mRNA. Inducible increase of HuR in murine innate compartments suppresses inflammatory responses in vivo. HuR over-expression induced the translational silencing of specific cytokine mRNAs, despite positive or nominal effects on their corresponding turnover. The present invention uses the fact that HuR acts in a pleiotropic fashion in inflammation through its functional interactions with specific mRNA subsets and negative posttransciptional modules.

The invention relates to the field of inflammation. The invention in particular relates to means and methods for modulating inflammation and inflammation type reactions of cells.

Innate immunity responds to infectious and immune stimuli, whilst preserving the balance between inflammation and tissue homeostasis. As the intricacies of innate receptors are being unveiled, it is becoming clear that their signals target selective gene expression modules for the prudent production of key inflammatory mediators. For example, the activation of Toll like receptors (TLRs) results in the NFkB-dependant transcription of numerous cytokines that define the inflammatory character of the innate response (Bonizzi & Karin 2004). This innate signature may then be modified by post-transcriptional mechanisms that adjust the thresholds of inflammatory protein synthesis. The engagement of the classical TLR4 pathway on macrophages triggers stress activated kinases which in turn modulate the stability and the translation of TNF, IL-1, IL-3, IL-6, IL-8 and Cox2 mRNAs (reviewed by Saldatvala, 2004). Recent evidence suggests that innate post-transcriptional control proceeds through the dynamic interactions of cis-mRNA elements residing in inflammatory mRNAs, like the AU-rich sequence motifs (AREs; Wilusz et al, 2001), and selective RNA binding proteins (RBPs). For example, Tristetraprolin (TTP) is a zinc finger containing protein that binds to the TNF, GM-CSF and IL-3 AREs in macrophages and promotes their de-adenylation and degradation (Carballo, 1998 & 2000). Similarly, the RBP TIA-1 binds to TNF and Cox2 AREs in macrophages and fibroblasts respectively, to inhibit their induced translational activation (Piecyk et al., 2000; Dixon et al., 2003). The significance of these interactions in inflammation has been exemplified in mouse systems with deficiencies in the TNF 3′ARE or the RBPs TTP and TIA-1, that are predisposed to chronic inflammatory pathologies (Taylor et al., 1996; Kontoyiannis, 1999; Piecyk et al., 2000).

Current hypotheses suggest that negative post-transcriptional regulators antagonize the functions of RBPs with positive effects on RNA metabolism, like the members of Elavl/Hu family (Chen et al, 2002; Keene & Tenenbaum, 2002). Elavl1/Hu-Antigen R (HuR) (also known as HuA) is the ubiquitously expressed member of this family with a prototypical RBP structure that includes two N-terminal RNA recognition motifs (RRM) with high affinity for a U-rich sequence (HuR Binding Motif, HBM), followed by a nucleocytoplasmic shuttling sequence and a C-terminal RRM recognising the poly-A tail (Fan & Steitz, 1998a, b; De Silanes et al., 2004). Although predominantly nuclear, HuR, shuttles between the nucleus and the cytoplasm acting as an RNA adaptor (Fan and Steitz, 1998b). Numerous studies have indicated that the cytoplasmic HuR can stabilize specific mRNAs (Brennan and Steitz, 2001) or variably affect their translational processing (Gallouzi et al, 2000; Kullmann et al., 2002; Mazan-Mamczarz et al 2003). However, the constitutive expression of HuR and the wide distribution of HBM among numerous ARE/non-ARE containing mRNAs, indicate that HuR recognition may not be very discriminative (De Silanes et al., 2004); hence the mechanisms involved in inducing the specificity of its functions remain elusive. With respect to inflammation, studies on macrophage cell lines suggested that innate sensitizers increase the cytoplasmic binding of HuR to cytokine mRNAs supporting their stabilization (Dean et al., 2001; Di Marco et al, 2001; McMullen et al, 2003; Cok et al, 2004). Furthermore, genetic approaches have identified mouse strains with mutations in the HBM of inflammatory mRNAs that correlate with the development of autoimmunity (Di Marco et al, 2001). These observations suggest that the overt upregulation of HuR could support the hyper-activation of inflammatory mediators to drive ensuing inflammation. However, the search for HuR's role in innate immunity has been obstructed by its putative involvement in central developmental processes (Levadoux-Martin et al., 2003), the difficulties in manipulating differentiated innate cells and the lack of tissue specific systems of permutation in vivo.

In the present invention it was found that HuR has regulates in inflammatory suppression and that it synergizes with negative post-transcriptional modulators to influence the biosynthesis of specific inflammatory mediators. The present invention therefore provides a method for modulating translation of a specific mRNA in a cell comprising modulating expression of a member of the Elavl1/Hu family of selective RNA binding proteins (RBPs) in said cell and incubating said cell. Said cell is incubated to allow transcription of said specific mRNA such that it may be affected by the modified presence of said member of the Elavl1/Hu family. In a preferred embodiment a method of the invention comprises reducing translation of a specific mRNA in a cell comprising over-expressing a member of the Elavl1/Hu family of selective RNA binding proteins (RBPs) in said cell and incubating said cell. Over-expression of said member in said cell result in a reduction in the biosynthesis of specific inflammatory mediators. Thus is a preferred embodiment of the invention a method of the invention wherein said member is over-expressed is used to dampen an immune response, preferably an inflammatory response. A method of the invention is therefore particularly suited for treating diseases that are associated with undesired inflammation symptoms.

The HuR/Elavl1 family is a class of RBP that have at least 90%, preferably at least 95% and more preferably at least 98% sequence identity on the amino acid level, with the human HuR protein and/or the mouse Elavl1 protein as depicted in FIG. 8. The members of said family preferably have a prototypical RBP structure that includes two N-terminal R-NA recognition motifs (RRM) with high affinity for a U-rich sequence (HuR Binding Motif, HBM), followed by a nucleocytoplasmic shuttling sequence and a C-terminal RRM recognising the poly-A tail. For the present invention it is preferred that the Elavl1/Hu family member is a mammalian HuR, preferably a human HuR.

A changed in translation is preferably measured by through measuring the amount of protein in a cell in relation to the amount of mRNA coding for said protein that is present in said cell. However, translation efficiencies can also be measured through other means. Such means is, for instance, determining the fraction of mRNA coding for the to be translated protein that is associated with polysomes.

The family of Elavl1/Hu RBP proteins bind to U-rich sequences called HuR Binding Motifs (HBMs). Thus in a preferred embodiment said specific mRNA comprise a HBM. The invention is particularly useful for modulating and preferably reducing translation of a collection of different mRNA in a cell, particularly a collection of different HBM containing mRNA. Thus in a preferred embodiment a method of the invention is provided wherein said specific mRNA comprises a binding site for said member. In a preferred embodiment said mRNA further comprises an ARE site. ARE sites are AU-rich elements in mRNA. In a preferred embodiment said ARE site is a type III ARE. Messengers containing a HBM and an ARE site are preferred as in conjunction with an ARE site, said member of the Elavl1/Hu family also stabilizes the messenger in a method of the invention. In a preferred embodiment said mRNA codes for a cytokine. Said preferably produces said cytokine in at least a part of its life or differentiation stage. In a preferred embodiment said mRNA comprises mRNA coding for TNFα, TNFβ, LTβ, IL-1, IL-2, IL-3, IL-8, IL-10, GM-CSF, G-CSF, M-CSF, Cox2, iNOS, TGFβ1, IFNα, IFNβ, IFNγ.

The member of the Elavl1/Hu family is preferably a mammalian member of the Elavl1/Hu family, preferably a primate, and more preferably a human primate member of the Elavl1/Hu family. The mammalian members are all conserved and exhibit the same function in kind in each mammalian cell, although the amount of function expressed may differ. Immune rejections, when used in vivo in a different species of mammal, are not likely because of their conserved amino acid sequence. Preferably, however, the cell wherein the expression of a member of the Elavl1/Hu family is modulated is of the same species as said member. Preferably, said cell a primate cell, preferably a human primate cell. Said cell is preferably an immune cell. Preferably a cell of the lymphoid and/or myeloid lineage. In a preferred embodiment said cell is a cell of the myeloid lineage. In a particularly preferred embodiment said cell is an antigen presenting cell, preferably a professional antigen presenting cell. Preferred cell types are dendritic cells, monocytes and/or macrophages.

Preferably said cell further expresses TIA. TIA-1 was found to share the cognate targets of HuR in macrophages and to require the TNF3′HBM/ARE for its association with the TNF mRNA. HuR and TIA-1 act synergistically towards the translational inhibition of TNF mRNA. In the absence of TIA-1, the translational silencing imposed by HuR over-expression on TNF mRNA was abolished, resulting in TNF protein overproduction and accordance with the HuR-induced accumulation of TNF mRNA. TIA-1 is also a principle component of mammalian stress granules (SG's) where mRNAs remain in a translationally silenced state destined for translation or degradation, in response to stress (Anderson and Kedersha, 2002). Under the same conditions, HuR co-localizes with TIA-1 in SGs (Stoecklin et al., 2004). Without being bound to theory it is postulated that since the accumulation of TIA-1 protein was not altered in transgenic cells, HuR aids the sequestration of TIA-1 onto the NF 3′ARE.

The expression of a member of the Elavl1/Hu family can be modulated in a various ways. Expression can be downregulated by providing the cell with specific complementary nucleic acid such as but not limited to antisense RNA, a specific miRNA, siRNA or other forms such hairpin structures wherein at least a part of said structure comprises said complementary nucleic acid. The complementary nucleic acid typically comprises RNA, although synthetic variants, or modifications thereof are also used such as locked nucleic acid, 2-O′-methyl modifications, morpholino's and other variants and modifications. The nucleotides in the nucleic acid may be naturally occurring nucleotides such as A, C, G, T or U but may also be synthetic variants or analogues thereof. A large variety of different analogues are available to the person skilled in the art. Of note, some analogue share the same base pairing properties as the nucleotide they are analogues to. Other have a broader base pairing capability and can pair with all purines, all pyrimidines or both. A complementary nucleic acid may be provided to the cell directly or may be produced from an expression cassette that is introduced into the cell. The expression of a member of the Elavl1/Hu family can be upregulated by providing the cell, for instance, with an expression cassette comprising a coding region for said member.

An expression cassette is typically provided to a cell by associating the nucleic acid with a gene delivery vehicle. Various gene delivery vehicles are available to the person skilled in the art. Preferred examples of such gene delivery vehicles are viral vectors and liposome vehicles. Preferred viral vectors are adenoviral vectors and adeno-associated viral vectors. Such vectors are very suited for local gene therapy purposes. The invention thus further provides a method for the treatment of local inflammation in a subject comprising locally providing said subject with a gene delivery vehicle comprising an expression cassette of the invention. Preferably, said expression cassette comprises a coding region for a member of the Elav1/Hu family. Thus in a preferred embodiment said cell is provided with a nucleic acid comprising a coding region for said member. Said gene delivery vehicle is preferably used for (locally) dampening an inflammatory response in a mammal, preferably an auto-immune inflammatory response. Preferably said gene delivery vehicle is used for the treatment of arthritis, preferably inflammatory arthritis, preferably rheumatoid arthritis. In another embodiment said gene delivery vehicle is used for the treatment of septic shock, inflammatory hepatitis, inflammatory bowel disease, cachexia, lung inflammation, graft versus host disease, host versus graft disease, multiple sclerosis or another auto-immune disease such as type I diabetes.

In another embodiment, the invention provides a method for the local stimulation of inflammation comprising providing a subject with an expression cassette of the invention. Preferably, said expression cassette comprises a transcription unit for producing RNA that is complementary to a member of the Elavl1/Hu family.

In a preferred embodiment said member comprises HuR or a functional part or derivative thereof. In one embodiment said member further comprises an additional stretch of amino acids. The additional stretch of amino acids can have various functions. In one embodiment said additional amino acids comprise a peptide tag. This enables easy detection and/or isolated of said member in or from said cell, respectively.

In another aspect is provided a method according to the invention, wherein said cell is a cell of a transgenic non-human mammal and wherein the cells of said non-human mammal comprises a heterologous nucleic acid sequence encoding said member of the Elavl1/Hu family. Further provided is a non-human mammal comprising a cell having integrated in its genome a heterologous nucleic acid sequence for over-expressing a member of the Elavl1/Hu family in said cell. Preferably said non-human mammal is a transgenic non-human mammal comprising said heterologous sequence in essentially every cell of the body.

A sequence is said to be heterologous when it is not naturally present in cells of said non-human mammal or when it is present in an altered position in the genome. Non-limiting examples of a heterologous nucleic acid sequence is a viral promoter, an endogenous promoter that is introduced in a different position than normal, an endogenous promoter that is mutated to express the associated coding region at a level or timing that is different from the normal expression pattern, a coding region for a member of the Elavl1/Hu family from a different species, or a coding region for a member of the Elavl1/Hu family that is introduced at a different genomic location. Said heterologous nucleic acid sequence preferably comprises a promoter that is operatively linked to a nucleic acid sequence encoding said member of the Elavl1/Hu family. In a preferred embodiment said heterologous nucleic acid sequence comprises a coding region for said member of the Elavl1/Hu family under transcriptional control of an inducible promoter. Said promoter preferably comprises Tet-repressor binding sites and wherein said cell further comprises a reverse tetracycline transactivator transgene (rTTA) (Urlinger et al., 2000; this reference is herein included by reference). Said heterologous nucleic acid sequence preferably allows over-expression of said member in a cell of the myeloid lineage of said mammal.

Said non-human mammal is preferably a rodent, preferably a mouse or a rat. Preferably said heterologous nucleic acid sequence encodes said member of the Elavl1/Hu family. Preferred is that said heterologous nucleic acid sequence comprises a coding region for said member of the Elavl1/Hu family under transcriptional control of an inducible promoter. Said promoter preferably comprises Tet-repressor binding sites and wherein said cell further comprises a reverse tetracycline transactivator transgene (rTTA) Further provided is a non-human mammal comprising a cell having integrated in its genome a heterologous nucleic acid sequence encoding a reverse tetracycline transactivator transgene (rTTA) operatively linked to a human lysozyme promoter (Clarke et al., 1996, this reference is herein included by reference).

In another aspect the invention provides a recombinant nucleic acid encoding a member of the Elavl1/Hu family under transcriptional control of an inducible promoter. Preferably wherein said inducible promoter comprises a Tet-repressor binding site.

The invention further provides a method for stabilizing specific mRNA in a cell comprising over-expressing a member of the Elavl1/Hu family of selective RNA binding proteins (RBPs) in said cell and incubating said cell to allow transcription of said specific mRNA. Preferably wherein said specific mRNA comprises a Hu-R binding motif and a type III ARE sequence.

In another embodiment the invention provides a method for modifying the rate of translation of specific mRNA in a cell comprising altering the level of a member of the Elavl1/Hu family in said cell.

### DETAILED DESCRIPTION

The invention is exemplified herein below by HuR as a member of the Elavl1/Hu family. The invention is not limited to this member. In the present invention the functions of HuR were analyzed in the modulation of inflammatory responses by means of its conditional over-expression: (a) in macrophage subpopulations; (b) in a temporally-restricted fashion for the direct assessment of rapid inflammatory gene expression programs; and (c) in direct correlation with pathogenic inflammatory responses in vivo. The N-terminally tagged HuR used, maintained all the characteristic features of the endogenous mHuR (nucleocytoplasmic localization and binding to ARE templates) and added to the total HuR activity. It was found that induced over-expression of HuR reduced both ex- and in-vivo inflammatory responses.

The effects of HuR over-expression was exemplified using a list of “inflammatory” mRNAs that is representative in terms of macrophage activation and putative HuR targets that contain different ARE structures. The analysis of these mRNAs demonstrated that HuR acts in a discriminative fashion targeting directly a specific mRNAs like those encoding TNF, Cox2 and TGFβ1 and affecting their biosynthesis at multiple yet different levels. In addition, HuR indirectly affected several gene expression modules through the regulation of transcriptional as exemplified in the case of TGFβ1, or other mechanism like in the case of IL-1β, that affect inflammatory gene expression. In addition, HuR functions are governed by the cis-elements in each independent mRNA species. For example, the TNF and Cox2 mRNAs contain an HBM next to a cluster III ARE and respond similarly to HuR in macrophages (increased stability, reduced translation) whereas the TGFβ1 mRNA does not contain AREs and HuR over-expression reduces its translation. These associations provide an answer to the seeming discrepancy between the wide distribution of the degenerate HBM in mRNA populations and the specificity of its functions (De Silanes et al., 2004). Additional evidence for this type of modulation is provided by the difference between the HBM and the binding sites of other ARE binding factors like AUF1 and TTP (Chen et al., 2002; Lal et al., 2004) as well as their different signalling requirements (Winzen et al., 2004). Inflammatory mRNAs can thus be clustered with respect to their UTR elements and their functional response to HuR.

HuR acts as a coordinator of downstream RBP associations that will be governed by the quantity and the intrinsic elements of a given mRNA subset, as well as the activation of specific RBPs in response to an external stimulus. This occurs, for instance, in macrophages. This is exemplified by our data on the modulation of TNF mRNA in macrophages. HuR binds to the TNF 3′HBM/ARE to increase TNF mRNA stability and reduce its translation in LPS stimulated macrophages, presumably by interfering with the functions of negative modulators like TIA-1 and TTP. TIA-1 was found to share the cognate targets of HuR in macrophages and to require the TNF3′HBM/ARE for its association with the TNF mRNA suggesting a synergy between HuR and TIA-1 towards the translational inhibition of TNF mRNA. In the absence of TIA-1, the translational silencing imposed by HuR overexpression on TNF mRNA was abolished, resulting in TNF protein overproduction and accordance with the HuR-induced accumulation of TNF mRNA. TIA-1 is also a principle component of mammalian stress granules (SG's) where mRNAs remain in a translationally silenced state destined for translation or degradation, in response to stress (Anderson and Kedersha, 2002). Under the same conditions, HuR co-localizes with TIA-1 in SGs further suggesting their putative interaction (Stoecklin et al., 2004). Since the accumulation of TIA-1 protein was not altered in transgenic cells, it may be that HuR aids the sequestration of TIA-1 onto the TNF 3′ARE.

In TTP deficient macrophages, the LPS induced pool of TNF mRNA increases as a consequence of increased mRNA stability (Carballo, 1998) and as demonstrated herein, HuR overexpression did not affect the TNF mRNA pool any further. In the presence of TTP, HuR over-expression induces the stabilization of TNF and Cox2 mRNAs, supporting a role of HuR in over-riding the TTP destabilization effect. Furthermore, prior evidence indicates the temporal basis of this antagonism. Firstly, the over-expression of TTP promotes the decay of HuR-stabilized TNF3′ARE reporters and TTP is sequestered into SGs that already contain HuR (Stoecklin et al., 2004). Secondly, LPS induces the accumulation of TTP whereas HuR is always in excess even prior to stimulation (Brooks et al., 2004; Cao et al., 2004 and our data). Finally, and as demonstrated herein, the overexpression of HuR reduces the translation of the TNF mRNA in TTP deficient macrophages. The reduction of TNF mRNA translation in this setting could result from the increased availability of stable TNF mRNA. Together, these observations show, that the cooperation between HuR and TIA-1 results in the formation of an “inert”—and in some cases stable—reservoir of mRNAs, which subsequently become labile for degradation by TTP.

The clinical significance of the current work is exemplified by the anti-inflammatory properties of the transgenic HuR in vivo. In the case of the LPS induced model of septic shock, HuR reduced the production of key inflammatory mediators. The anti-inflammatory properties of HuR overexpression was indicated in the context of inflammatory hepatitis. The nature of the exudate infiltrates and the actual response of hepatocytes, shows a correlation between the reduction of TNF that is known to induce hepatocyte apoptosis (Sass et al., 2002) and attenuated liver damage in the context of HuR overexpression in macrophages. It is interesting to note that hepatitis-inhibiting cytokines like IL-6 (Sun et al., 2004), remained unaffected indicating that beneficial responses could be maintained in the context of HuR overexpression. These observations indicate that strategies aiming in the modulation of HuR have a potential clinical benefit.

### BRIEF DESCRIPTION OF THE DRAWINGS

FIG. 1. Induction of HuR overexpression in transgenic macrophages and myeloid-rich tissues. (A) Diagrammatic representation of the binary transgenic system. (B) Immunodetection of the HA-tagged and total HuR in Tg632 & Tg662 TEPM extracts stimulated with increasing Dox concentrations (μg/ml). N: non-transgenic extracts; TCE: control extracts from 293T cells transfected with an expression vector for HA-HuR. Actin is shown as a quantitative control. (C) Flow cytometric detection of total and HA-HuR protein following intracellular staining of transgenic myeloid (CD11b⁺) and non-myeloid (CD11b⁻) splenocytes, lung exudate cells, peripheral blood monocytes (PBL) and peritoneal cavity cells (PEC), after the in vivo administration of Dox (open diagram) or sucrose diluent (closed diagram) for 14 days. The dashed lines indicate background staining with isotype control antisera. (D) Immunodetection of HuR protein, plus/minus Dox and/or LPS (2 hrs) in nuclear (N) and cytoplasmic (C) extracts from Tg632 TEPM. U1snRNP and α-Tubulin are shown as markers of N/C separation. (E) R-EMSA with cytoplasmic extracts from non-transgenic (NTMΦ) and Tg632 (TgMΦ) TEPM, plus/minus Dox and/or LPS as in D. Binding reactions using a probe containing the 3′HBM/ARE of TNF mRNA yielding 3 complexes (c1-c3); Note that Dox increases total binding in transgenic extracts even in the absence of LPS (lane 2); (F) Shift assays following incubation of transgenic extracts with anti-HA, -HuR and control antisera (Ig). Note that Dox induces the presence of a HuR (s2) supershifted complex (lane 8) in the absence of LPS. In LPS-treated transgenic extracts, Dox induced the presence of the lower mobility HA complex (s1-lane 6) concomitant to a change in the level and mobility of the HuR supershifted complexes (s2-lane 4)

FIG. 2. Differential effects of HuR on proinflammatory cytokine biosynthesis. (A) Representative Northern slot blots for the detection of TNF, IL-1β, IL-6. TGFβ1, Cox2 and actin mRNAs in Tg632 (T) and control (N) TEPM following stimulation with increasing Dox concentrations (0-10 μg/ml) and LPS (2 hrs). (B) Graphical correlation between the LPS induced accumulation of TNF, IL-1β, Cox2 and TGFβ1 mRNAs () and proteins (◯) in Dox-induced Tg632 macrophages. mRNA values were derived from the densitometric quantitation of the corresponding northern blots (n=3 experiments with TEPM pools from 3-5 mice/group/exp.). Protein values were derived from the analysis of culture supernatants or macrophage extracts following stimulation with Dox and LPS (12 hrs) using specific ELISAs or immunoblots, from individual cultures (n=5-7 mice/group). Results shown as percentages of wild type control values (mean±SD). (*, **) denote statistically different values (p<0.01) in protein and mRNA values respectively. (C) Semi-quantitative RT-PCR for the detection of selected mRNAs immunoprecipitating with total or HA-HuR in RIP assays performed using transgenic TEPMs stimulated with LPS (2 hrs) plus/minus Dox; RIP assays with control antisera and total inputs are also indicated. (D) Western blot of the IP material for the detection of the endogenous (mHuR) or the transgenic (HAHuR). The heavy (HC) and light chains (LC) of the antibodies are indicated.

FIG. 3. Differential effects of HuR on cytokine mRNA stability and transcription. Mouse TEPM were stimulated with Dox and LPS or LPS alone to induce HuR and cytokine biosynthesis; Decay analysis. Northern analysis of TNF and Cox2 (A) as well as of TGFβ1 and GAPDH (B) mRNAs from Tg632 and control, untreated (T, N) or Dox treated (TD, ND) TEPM stimulated with LPS (2 hrs) and then with actinomycin D (10 μg/ml); semi-logarithmic plots of data (A) from the densitometric analysis of the northern blots for TNF and Cox2 mRNAs normalized to GAPDH. The half-lives of the corresponding mRNAs from Tg632 TEPM in the presence (Dt½) or absence (t½) of Dox are indicated. (C) Analysis of TGFβ1 transcription. Nuclei run-on experiment with labelled RNAs from untreated or Dox-treated Tg632 TEPM, following LPS stimulation as before and hybridized with limited dilutions (μg) of TGFβ1 cDNA (P) spotted onto nylon filters; Bar graph shows results (mean±SD) from 3 experiments (TEPM pools from 2-3 mice/group/exp.); (*) p<0.05

FIG. 4. Differential effects of HuR on cytokine mRNA translation. Polysome analysis in untreated or Dox treated Tg632 TEPMs, stimulated with LPS (2 hrs) and subsequent separation of cytoplasmic lysates in sucrose gradients. Upper left panel-Representative profiles of RNA samples in selected fractions (1-15) on agarose gels and of the 260 nm UV absorption across all of the eluted fractions indicating the peaks corresponding to free RNA (F), monosomal (40S, 60S, 80S) and polysomal fractions. Other Panels—Densitometric quantitation of TNF, Cox2, TGFβ1, IL6 and β-actin RNAs in individual fractions was determined following hybridization and autoradiography. The dashed line indicates the separation between monosomal and polysomal fractions; (o) LPS; () Dox+LPS. Representative dot-blots of the hybridized fractions are presented. The number of polysome fractions from independent experiments (mean±SD; n=3-5 experiments with TEPM pools from 3-5 mice/group/exp.) containing the indicated RNA and the values denoting statistical differences (p) are shown.

FIG. 5. The myeloid restricted overexpression of HuR inhibits inflammatory responses in vivo. Tg632 and control mice were fed ad libidum with Dox (+D) or sucrose diluent (+S) for a period of 3 weeks. (A) Serum levels of TNF, IL1β, IL6 and TGFβ1 protein, 90 min post LPS administration (200 μg) in Tg632 and littermate control (N) mice. Bar graphs show individual and mean values (±SD) following analysis with the corresponding ELISAs. (*) p<0.1 relative to non-treated transgenic animals. (B, C) Histologic examination of transgenic livers 48 hrs past the administration of ConA (30 mg/Kgr). Hematoxylin-eosin staining of paraffin-embedded liver sections from sucrose (B) and Dox (C) treated groups indicating the complete absence of inflammation and hepatocyte damage in HuR overexpressing animals. (D) Serum AST, TNF, IL6 and TGFβ1 levels measured 24 hrs post ConA administration. Bar graphs showing individual and mean values (±SD) following analysis with the corresponding ELISAs or bioactivity assays. (*) p<0.005 relative to non-treated transgenic animals. (**) p<0.05 denotes the increase in TGFβ1 levels relative to controls. Annotations: (o) control mice fed with Sucrose; (∇) control mice fed with Dox; (□) Tg632 mice fed with Sucrose; (⋄) Tg632 mice fed with Dox.

FIG. 6. HuR requires the TNF3′HBM/ARE to modulate TNF biosynthesis: correlations with ARE binding proteins. (A) Northern slot blots for the detection of TNF mRNA (T) with (+) or without (Δ) the TNF 3′HBM/ARE in Dox and LPS stimulated Tg632⁺Tnf^{-/-} and Tg632⁺Tnf^{ΔARE/-} BMDM. GAPDH mRNA (G) is shown as loading control. (B) Levels of secreted TNF protein from Tg632⁺Tnf^{+/-}, Tnf^{ΔARE/-} and Tg632⁺Tnf^{ΔARE/-} BMDM stimulated with LPS (12 hrs) plus/minus Dox. Data shown as individual and mean values (±SEM) from 6-8 mice group. (*) p<0.05. (C, D) Polysomal distribution of the TNF^{ΔARE} and actin mRNAs in LPS treated Tnf^{ΔARE/-}, and Dox induced/uniduced Tg632⁺Tnf^{ΔARE/-} BMDM cultures (n=2 experiments with 3-5 mice/group/exp.). Representative densitometric quantitation of actin (C) and TNF (D) mRNAs in individual fractions determined from the analysis of dot blots as before. (E) Semi-quantitative RT-PCR for the detection of selected mRNAs immunoprecipitating with HuR or TIA-1 in RIP assays performed using Tnf^{ΔARE}/^{ΔARE}TnfRI^{-/-} or Tnf^{+/+}TnfRI^{-/-} BMDM cultures, stimulated with LPS (2 hrs); RIP assays with control antisera and total inputs are also indicated. (F) Western blot of the IP material for the detection of mouse TIA-1. The heavy (HC) and light chains (LC) of the antibodies are indicated. (G) Western blot analysis of Tg632⁺ BMDM extracts for the detection of HuR, TIA-1 (actin as a loading control) and TTP (using ponceau staining as a loading control) in the presence or absence of Dox and LPS.

FIG. 7. Genetic and functional interactions between HuR, TIA-1 and TTP towards the modulation of TNF biosynthesis. (A) TNF protein levels in culture supernatants from LPS stimulated Tg632⁺, TTP^{-/-}, TIA^{-/-}, Tg632⁺TTP^{-/-}, Tg632⁺TIAI^{-/-} BMDM for 12 hrs, in the presence (black bars) or absence (grey bars) of Dox. Data shown as mean values ±SEM from 6-8 mice per group. (p) denote values of statistical comparison. (B) Polysomal distribution of the TNF mRNA in LPS treated TTP^{-/-} and Dox treated/untreated Tg632⁺TTP^{-/-} BMDM. Representative densitometric quantitation of TNF mRNA in individual fractions following hybridization and autoradiography as before. Indicated values from 3 independent experiments with cultures from 2-3 mice/group.exp. P denotes significant difference. (C) Representative northern blot analysis of TNF mRNA accumulation in Tg632⁺, TTP^{-/-}, TIA^{-/-}, Tg632⁺TTP^{-/-}, Tg632⁺TIA1^{-/-} BMDM following LPS stimulation for 0, 2 and 6 hrs. GAPDH mRNA is shown as loading control. (D) Polysomal distribution of the TNF mRNA in LPS treated TIA^{-/-} and Dox treated/untreated Tg632⁺ and Tg632⁺TIA1^{-/-} BMDM. Representative densitometric quantitation of TNF mRNA in individual fractions following hybridization and autoradiography from n=2 experiments with 4-5 mice/group/exp. (E) Semi-quantitative RT-PCR for the detection of selected mRNAs immunoprecipitating with HuR or TIA-1 in RIP assays performed using Tg632 BMDM stimulated with LPS, plus/minus Dox. RIP assays with control antisera and total inputs are also indicated. (F) Supershift assays with cytoplasmic extracts from LPS-stimulated Tg632 BMDM in the presence or absence of Dox using a specific probe as before and following incubation with anti-HuR, -TIA1 and control antisera (Ig). Note the presence of the 3 binding (c1-c3) and the anti-HuR/TIA-1 supershifted complexes (s).

FIG. 8. Amino acid sequence of human HuR and comparison to mouse homologue Elavl1. Protein sequences obtained EnsEMBL with Peptide Id numbers ENSP00000264073 for human and ENSMUSP00000045140 for mouse forms respectively. Proteins were aligned using an AlignX algorithm. Calculated identity is 98.2%

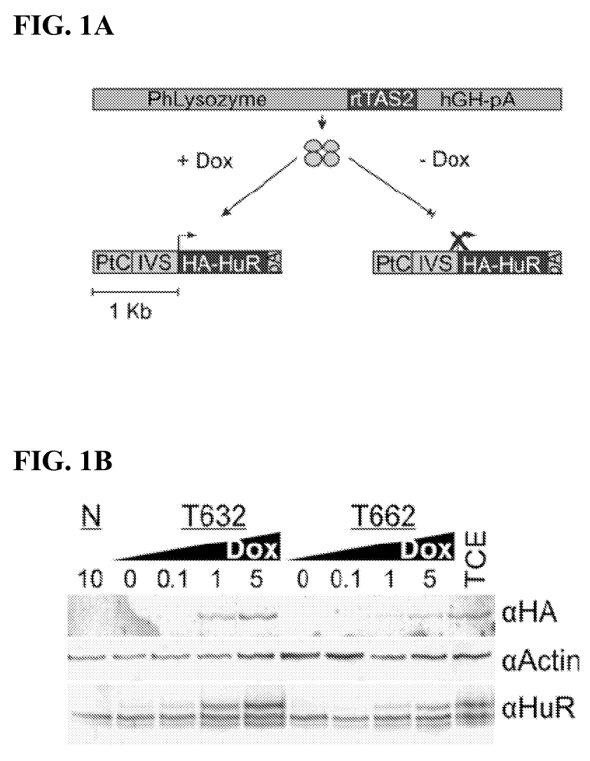

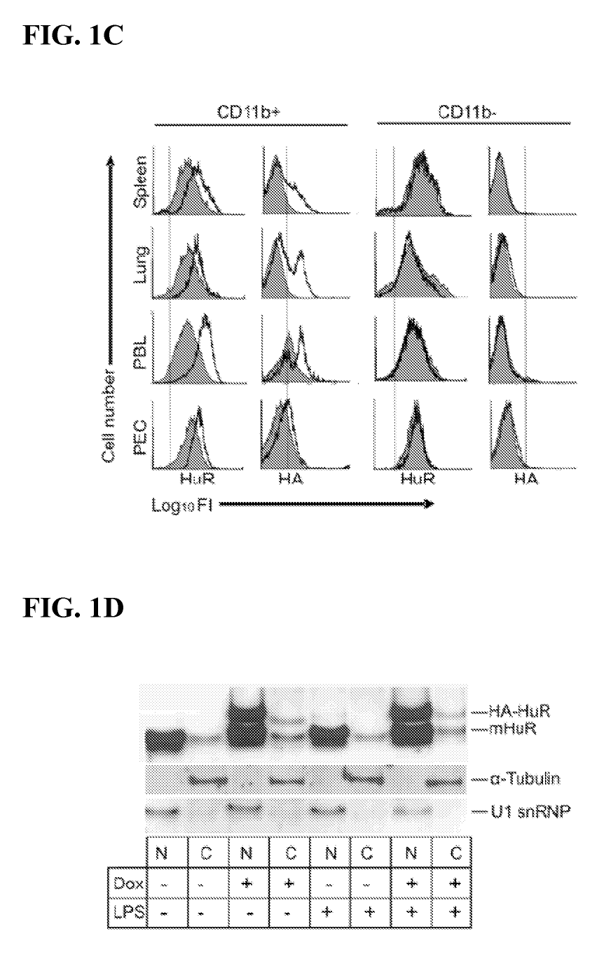

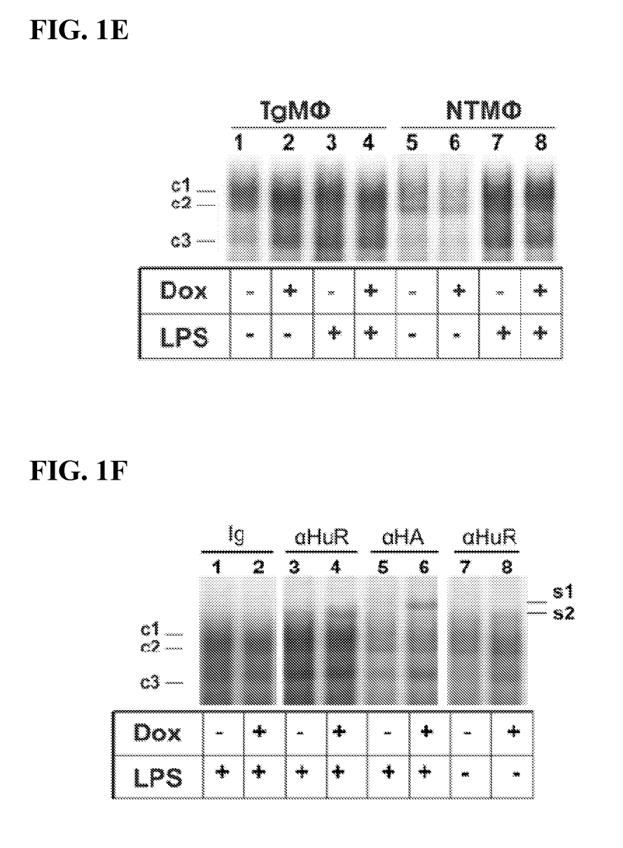

Sup. FIG. 1. (A) Immunoblots for the detection of recombinant GST-HuR and GST-HA-HuR proteins in total bacterial extracts (TE) and eluted purified fractions (EL) using anti HA and anti HuR antibodies. Ponceau S (PonS) staining is shown as a protein loading control. (B) R-EMSA using specific ³²P-labeled riboprobe (SP) containing the TNF 3′HBM/ARE incubated with the indicated concentrations of GST-HuR, GST-HAHuR or GST; note the gradual appearance of a low and a high mobility complex (c1-c2). The specificity of protein binding was assessed in competition assays by adding an excess amount of cold probe (comp). (C) Protein titration curves for complex C1 ( HuR and ◯ HA-HuR) and complex C2 (□HuR and ∇ HA-HuR) indicating the similarities in binding. Values are relative numbers of a phosphorimager reading of the bound RNA from different protein concentrations used in (B). (D) R-EMSA with the recombinant proteins GST-HuR and GST-HAHuR using increasing amounts of both specific (SP) and mutant (MP) ³²P-labeled riboprobes to assess specificity and relative affinities. (E) Determination of active protein concentration. RNA titration curves for complex C1 ( HuR and ◯ HA-HuR) and complex C2 (□HuR and ∇ HA-HuR) showing equal binding activity of GST-HuR and GST-HA-HuR. Values are relative numbers of a phosphorimager reading of the amount of RNA in the shifted bands in (D). (F) Assessing the specificity of binding in transgenic macrophage extracts. R-EMSA with cytoplasmic extracts from Tg632 (Tg) and wild type (NT) macrophages; protein extracts were incubated with the specific probe containing the 3′HBM/ARE of TNF mRNA (SP) yielding three complexes (c1, c2, c3). The specificity of the protein binding is shown by using the mutant (MP) ³²P-labeled riboprobe or in competition assays with cold riboprobes (SP_{cold}, MP_{cold}). (G) Primary sequence for the transcribed human TNF HBM/ARE (GeneBank# NM000549) containing riboprobes used in this study. The specific probe (SP) contains the natural sequence whereas in the mutant probe (MP) every UU or UUU has been replaced by UG and GGU respectively.

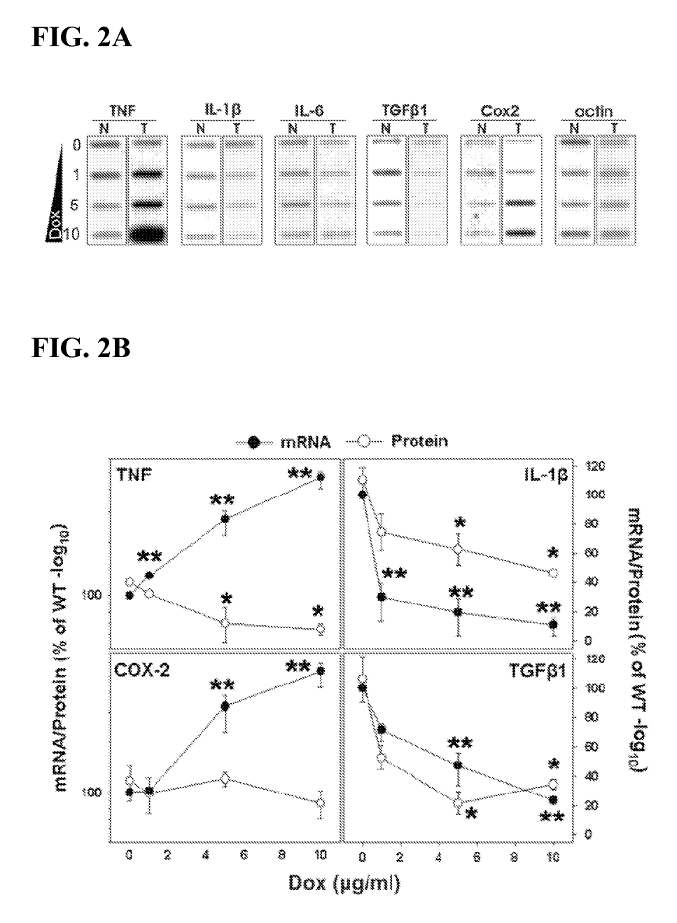

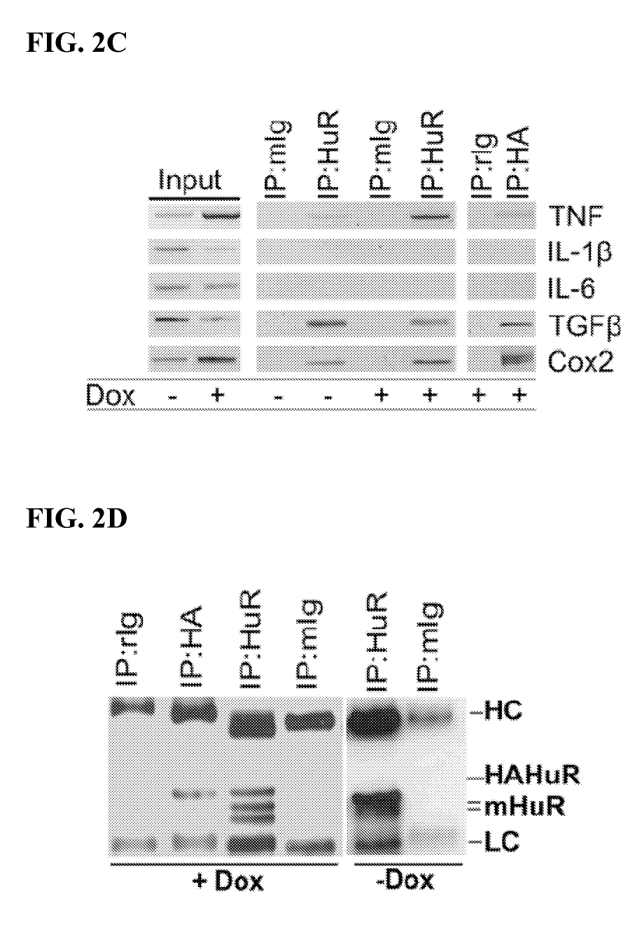

Sup FIG. 2. Selective interactions among the RNA binding proteins HuR and TIA-1 and specific cytokine mRNAs in RAW264.7 macrophages. (A) Semi-quantitative RT-PCR for the detection of selected mRNAs immunoprecipitating with HuR or TIA-1 in RIP assays performed using Raw 264.7 macrophages plus/minus LPS. RIP assays with control antisera and total inputs are also indicated. Note that IL6 mRNA co-immunoprecipitates with HuR but not with TIA-1.

### EXAMPLES

### Results

### Inducible Over-Expression of HuR in Transgenic Macrophages and Tissues.

We generated a binary mouse transgenic system through the co-injection of (i) an “effector” reverse tetracycline transactivator transgene (rtTA) controlled by the human lysozyme promoter and (ii) a “target” HA-tagged human HuR transgene controlled by a Tet-operator containing promoter (FIG. 1A). rtTA transcripts were detected in ex vivo cultures of macrophages as well as in myeloid-rich tissues from two transgenic lines with 5 (Tg632) or 2 (Tg662) copies of the rtTA and comparable (6-8) copies of the HA-HuR transgenes (not shown); marginal HA-HuR transcripts were detected in resting mouse macrophages and tissues, but transgenic HuR protein was undetectable in the corresponding protein extracts (FIG. 1B and not shown). The inducibility of the system was assessed through the stimulation of elicited (TEPM) and bone marrow derived (BMDM) macrophage cultures, with Doxycycline (Dox) (see also Experimental Procedures). Tg632 macrophages displayed the highest induction profile that yielded a 2.5-3 fold increase in total HuR protein at 5-10 μg/ml of Dox, peaking at 20 hrs post stimulation and remaining constant thereafter (FIG. 1B and not shown). The induction of HA-HuR did not affect the accumulation of mouse HuR (mHuR), although an increase in the lower anti-HuR reacting-band was noted (FIG. 1B). In vivo, the maximal and more widespread expression of HA-HuR was achieved in Tg632 mice, following the oral administration of 2 mg/ml Dox, for 15-21 days. Flow cytometric analysis of leukocytes derived from different tissues of induced Tg632 mice indicated the expression of HA-HuR in CD11b⁺ monocytes/macrophages but not in CD11b⁺Gr1⁺ granulocytes nor CD11b⁻ non-myeloid cells (FIG. 1C and not shown).

Next we examined the distribution of HuR in nuclear (N) and cytoplasmic (C) extracts from transgenic TEPM, as an indirect measure of its shuttling functions. In control TEPM, mHuR distributed at a 5:1 N/C ratio, which was not altered in the presence of LPS (FIG. 1D). In transgenic TEPM, the induction of HA-HuR increased the total HuR content in both compartments but did not affect the total N/C ratio suggesting that: (a) transgenic HuR shuttles in a fashion similar to the endogenous; and (b) the increase in HuR levels does not affect its N/C distribution. Next, we tested cytoplasmic extracts from control and induced Tg632 TEPM for binding to an RNA probe encompassing the 3′HBM/ARE of the human TNF mRNA (Supplemental FIG. 1). Three specific complexes (c1, 2 & 3) were readily observed in all reactions and did not appear in the presence of a mutant probe (FIG. 1E & Supplemental FIG. 1). LPS increased primarily c1 and c3 in control extracts whereas in induced transgenic extracts, these 2 complexes were increased even in the absence of LPS. Similarly, analysis of recombinant HuR and HA-HuR proteins indicated their similar affinities for the target sequence yielding 2 complexes (Supplemental FIG. 1). Incubation of induced transgenic extracts with specific antisera, demonstrated the increase in total and HA-HuR binding as indicated by the corresponding supershifts (s1-2; FIG. 1F). However, the presence of these antisera did not obliterate the signals corresponding to c1 & 3, suggesting that HuR comprises a fraction of the proteins in these complexes (FIG. 1F). Thus HA-HuR shares the binding capacity of mHuR and the total HuR activity is increased in induced transgenic macrophages.

### Effects of HuR Overexpression on Cytokine Biosynthesis

To examine the effect of HuR overexpression on the biosynthesis of inflammatory mediators, we analyzed the accumulation of 27 representative inflammatory mRNAs in induced Tg 632/662 and control TEPM using ribonuclease protection assays (RPAs) and northern slot-blots; 15 of those mRNAs contained variable ARE motifs (types I-V) according to the ARED database (http://rc.kfshrc.edu.sa/bssc/ARED_GENE). In total, 16/27 mRNAs were expressed in LPS-stimulated TEPMs and 4 were significantly affected in induced transgenic cultures relative to controls (Supplementary Table 1). Specifically, and only following LPS stimulation, the levels of TNF and Cox2 mRNAs were increased whereas the corresponding levels for IL-1β and TGFβ1 mRNAs were reduced. These changes were further validated in Tg632 TEPM induced with a range of Dox concentrations using IL-6 (inducible) and β-actin (constitutive) mRNAs as controls. In all cases, the effects observed were proportional to the concentration of Dox used and were not detected in Dox treated wild type controls (FIG. 2A, B). Similarly, the levels of secreted TGFβ1 and IL-1β proteins were reduced in induced Tg632 cultures, whereas the levels of secreted IL-6 remained unaffected (FIG. 2B and not shown). Quite surprisingly, secreted TNF protein was reduced in these cultures and intracellular Cox2 protein was not altered (FIG. 2B), despite the increase in their mRNA pools.

Ribonucleoprotein Immunoprecipitation (R-IP) assays were performed to assess whether the affected mRNAs interacted with HuR in macrophages. Due to the increased cellular requirement of these assays, HuR and control IPs were initially performed in cytoplasmic extracts from the macrophage line RAW 264.7, to assess the LPS induced interactions between HuR and target mRNAs (Supplemental FIG. 2); subsequently these interactions were validated in induced and LPS stimulated Tg632 macrophages following IPs with anti-HuR, -HA or control antisera (FIG. 2C, D). In all cases, IP-associated RNA was reverse transcribed for the detection of the selected transcripts via semi-quantitative PCR. TNF mRNA associated with HuR in RAW264.7 in an LPS inducible fashion whereas the Cox2 and TGFβ1 mRNAs were constitutively present in the JPs despite the induced increase of their mRNA pools (Supplemental FIG. 2). Interestingly, an association between HuR and the IL-1β mRNA was not detected. A similar picture emerged following the IP of total and HA-HuR from induced Tg632 macrophages, suggesting that the transgenic HuR may directly affect the biosynthesis of TNF, TGFβ1 and Cox-2 but it is not directly inhibiting the biosynthesis of IL-1β (FIG. 2C). Finally, the IL-6 mRNA was associated with HuR in RAW264.7, but not in transgenic and control TEPM indicating a discrepancy between this cell line and primary cultures (FIG. 2C).

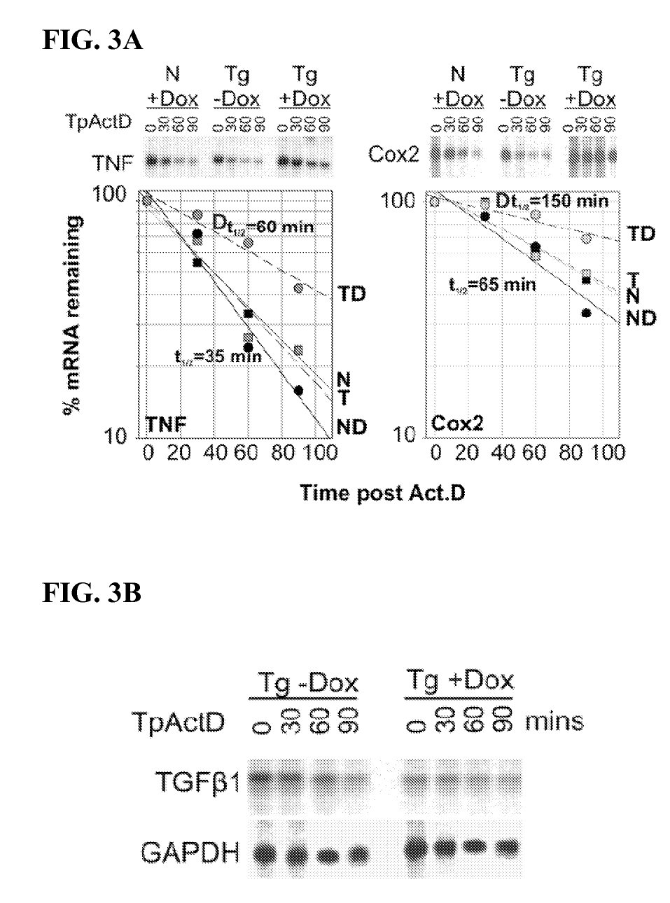

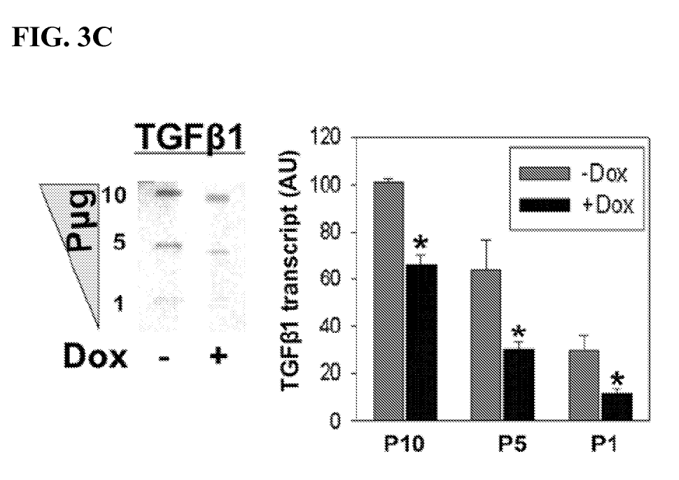

Next we examined the decay of the selected HuR targets in transgenic macrophages, following transcriptional blockade with Actinomycin D. Northern analysis of remnant mRNA levels indicated significant increases in the half lives of TNF and Cox2 mRNAs, but not in TGFβ1 mRNA, in induced transgenic TEPMs (FIG. 3A, B). Nuclei-run on analysis indicated a 40% reduction in the levels of TGFβ1 transcript produced in induced transgenic cultures, whereas the transcriptional output of TNF and Cox2 genes was not altered (FIG. 3C and not shown). Together, the data show that HuR induced the stabilization of TNF and Cox2 mRNAs but reduced the production of TGFβ1 mRNA through a mechanism affecting its transcription.

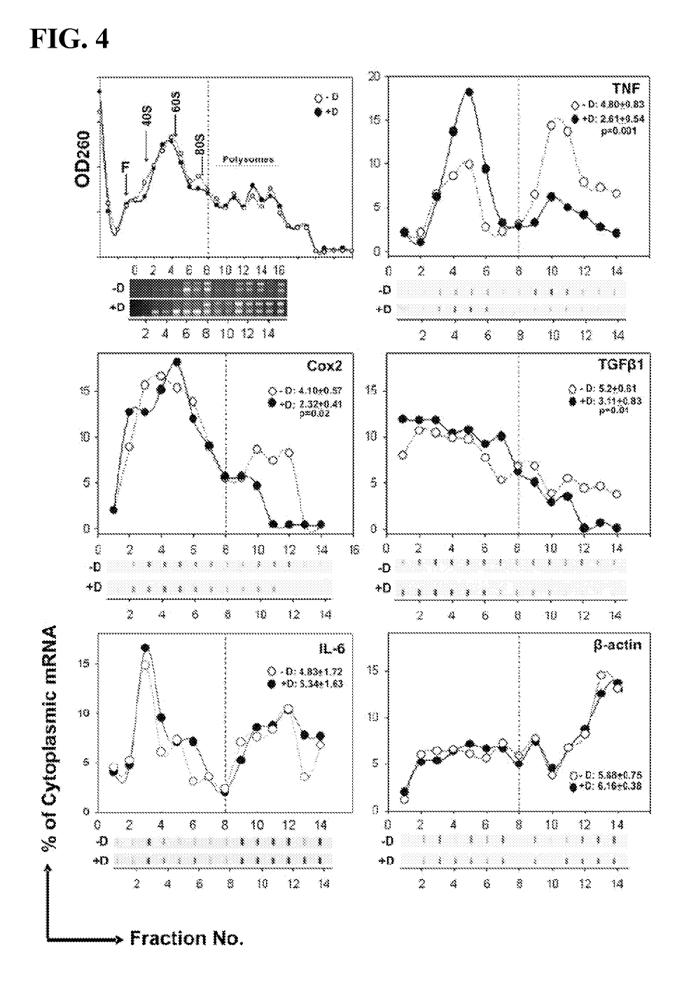

To understand the basis of the HuR-induced discordance between target mRNA accumulation and translational output, we analyzed the polysomal distribution of these mRNAs in transgenic macrophages (FIG. 4). The polysomal fractions from induced and LPS-stimulated transgenic TEPM displayed a clear reduction in the levels of TNF, Cox2 and TGFβ1 mRNAs relative to the uninduced control cultures; IL6 and β-actin mRNAs were similarly distributed in polysomes from induced and control TEPM, indicating that HuR specifically affects the translation of TNF, Cox2 and TGFβ1 mRNAs in LPS stimulated macrophages.

It is noteworthy that the similarity of the TNF and Cox2 mRNAs in response to HuR overexpression (i.e. increased stability, reduced translation) correlates with similarities in their 3′UTRs since they both contain HBMs and Type III AREs (Supplementary Table 1 & Data Set). In contrast, the TGFβ1 mRNA contains only HBM-like sequences and the effect of HuR overexpression is restricted to translational suppression. Taken together, these results demonstrate that in macrophages, HuR (a) targets specific inflammatory mRNAs; (b) its overexpression can suppress the translation of selected inflammatory mRNAs despite differential effects on mRNA stability; and (c) differences in HuR-instigated responses could correlate with different 3′UTR signatures.

### HuR Overexpression Inhibits Inflammatory Responses In Vivo

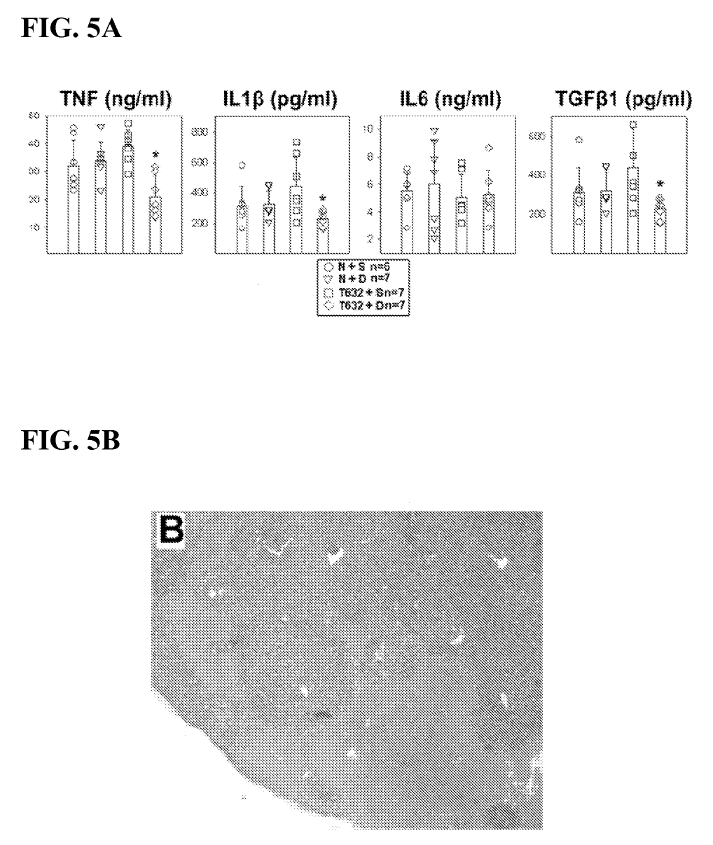

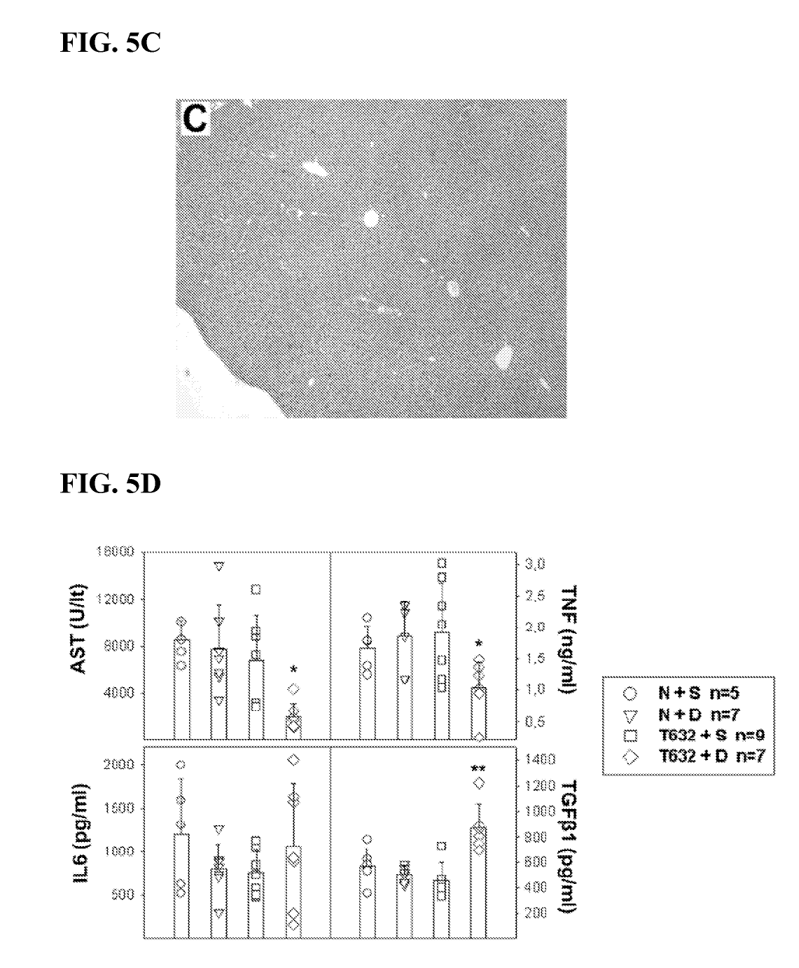

To relate with the ex vivo data, transgenic and littermate control mice were fed with Dox to induce the HA-HuR and were subsequently injected with LPS to analyze cytokine production in vivo. As can be seen in FIG. 5A, the in vivo induction of transgenic HuR, significantly reduced the levels of TNF, IL-1β and TGFβ1 in the sera of the LPS-challenged Tg632 mice but was not sufficient to alter their sensitivity to LPS-induced shock (Supplementary Table 2). To examine further the anti-inflammatory effects of HuR over-expression, we switched to a more restricted model of inflammatory degeneration. The peripheral administration of Concanavalin A (ConA) in the mouse induces inflammatory hepatitis as a consequence of an adverse lymphocytic reaction that in turn activates innate cells to release inflammatory mediators like TNF that cause hepatocyte death (Sass et al., 2002). Induced and un-induced, transgenic and littermate control mice were injected with ConA and the extent of hepatitis was assessed through the release of hepatic transaminases in the periphery. Following the administration of ConA, plasma aspartate aminotransferase (AST) levels were significantly increased in all control groups but appeared only marginally elevated in induced Tg632 mice (FIG. 5D). Similarly, the levels of TNF protein in the sera of induced Tg632 mice were significantly lower to control levels, whereas serum IL-6 was not significantly altered. Serum TGFβ₁ was significantly elevated in the induced Tg632 mice (FIG. 5D) which could result from its release by hepatocytes as a counter-response to hepatic assault (Sass et al., 2002) and not by macrophages. Histopathological evaluation of liver sections from ConA-treated mice indicated the consistent absence of inflammatory exudates and hepatocyte damage in the induced Tg632 liver samples (FIG. 5B, C). Overall, our data demonstrate that the myeloid restricted overexpression of HuR in vivo suppresses inflammatory responses and reduces the production of key inflammatory mediators.

Functional Interactions between HuR and Negative Post-Transcriptional Modulators

The differential modulation of cytokine mRNAs by HuR, suggested that its effects relate to its interactions with selected mRNA elements, and/or other downstream RBPs with more defined properties. To examine this hypothesis, we focused on the TNF mRNA and its 3′HBM/ARE that (a) modulates mRNA stability and translation in macrophages and (b) is negatively regulated by TTP and TIA-1.

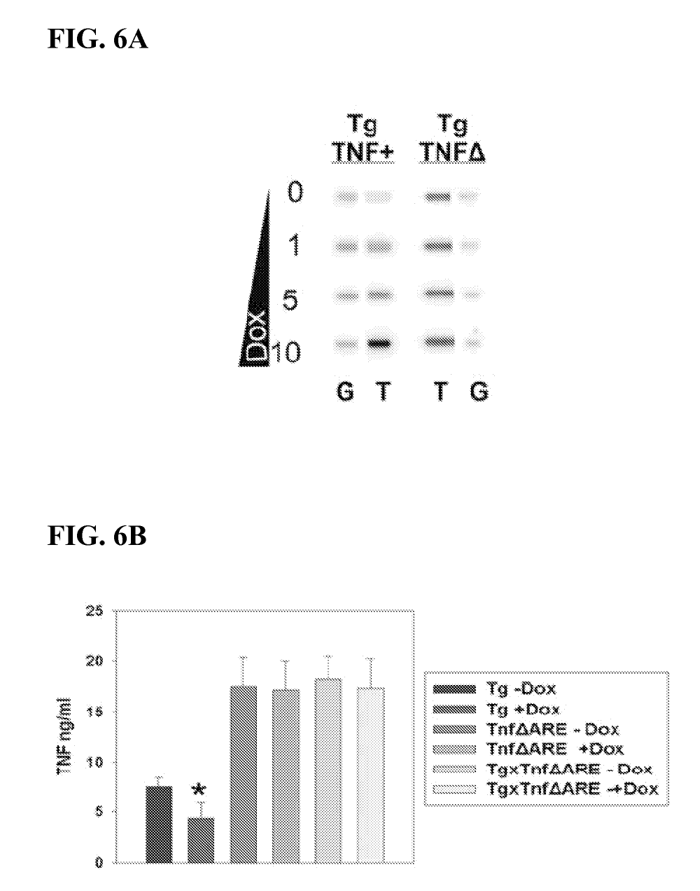

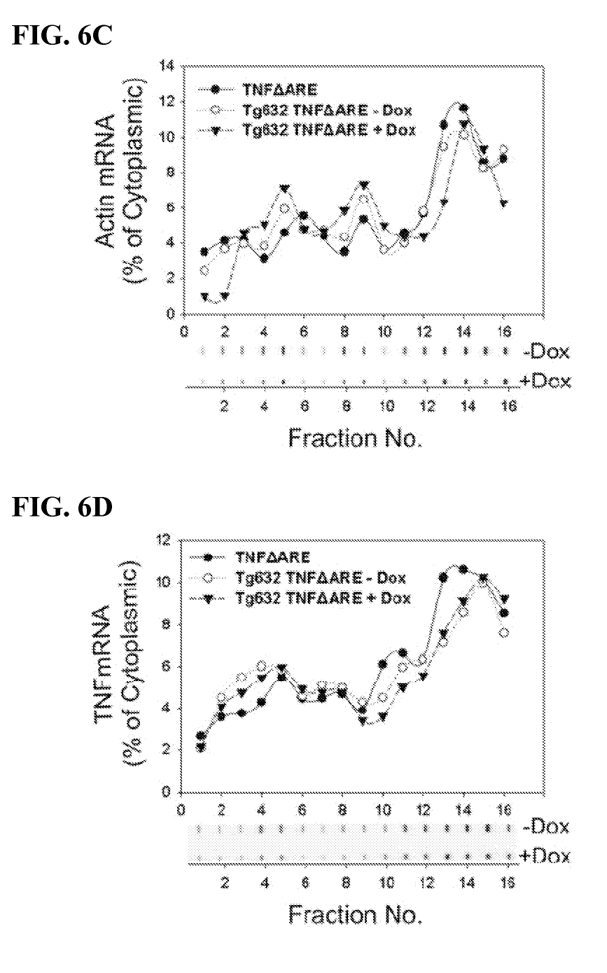

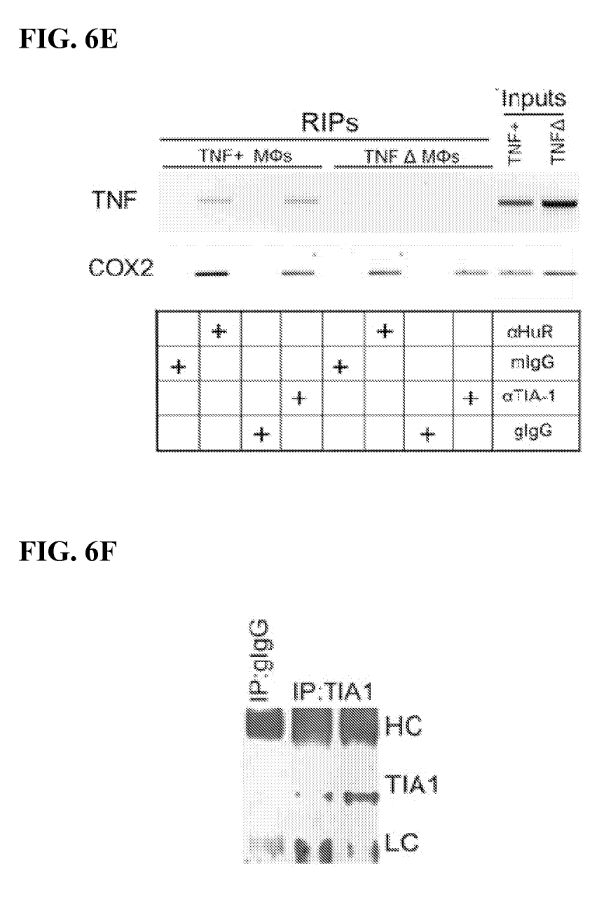

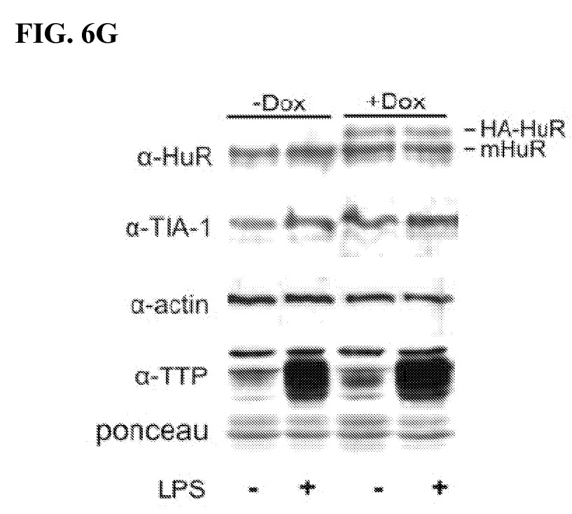

To address whether HuR requires the TNF3′HBM/ARE to affect the fate of TNF mRNA in macrophages, the Tg632 HuR allele was introduced in mutant Tnf^{ΔARE} mice bearing a targeted deletion of this element (Supplementary data set). Our analysis was restricted to a monoallelic TNF system (Tnf^{ΔARE/-}) of in vitro differentiated BMDMs to avoid the inflammatory effects developing in homozygotic Tnf^{ΔARE/ΔARE} mice (Kontoyiannis et al., 1999). LPS-stimulated Tnf^{ΔARE/-} BMDM accumulate 2-3 fold higher levels of mutant TNF mRNA and TNF protein than Tnf^{+/-} controls due to its increased stability and constitutive translation (Kontoyiannis et al., 1999; 2001). Dox did not alter these parameters in Tnf^{ΔARE/-} BMDM, but affected the levels of TNF mRNA and protein in the Tg632⁺Tnf^{+/-} BMDM cultures as before (FIG. 6A-D and not shown). However, in Tg632⁺ Tnf^{ΔARE/-} BMDM, the induction of transgenic HuR did not affect the accumulation, the polysome association and the protein output of TNF^{ΔARE} mRNA indicating that HuR could not affect the stability/translation of TNF mRNA in the absence of the TNF3′HBM/ARE (FIG. 6A-D). The possibility of saturation in TNF biosynthesis in our assay was excluded since homozygotic Tnf^{ΔARE/ΔARE} BMDM produce twice the amount of TNF mRNA and protein than Tnf^{ΔARE/-} BMDM (Kontoyiannis et al., 1999). Finally, RIP assays were performed to assess whether the TNF^{ΔARE} mRNA interacts with HuR in macrophages. To enhance the clarity of our observations we utilized BMDMs derived from homozygotic TNF^{ΔARE/ΔARE} that are devoid of TNF receptor I and thus do not develop inflammatory disease (Kontoyiannis et al., 1999). As can be seen in FIG. 6E, the TNF mRNA associated with HuR in RIPs from Tnf^{+/+}TnfRI^{-/-} BMDM but not from Tnf^{ΔARE/ΔARE}TnfRI^{-/-} BMDM, whereas Cox2 mRNA was detected in all HuR-IPs. Overall our results demonstrate that HuR requires the presence of the 3′HBM/ARE to interact with the TNF mRNA and affect its translation and turnover.

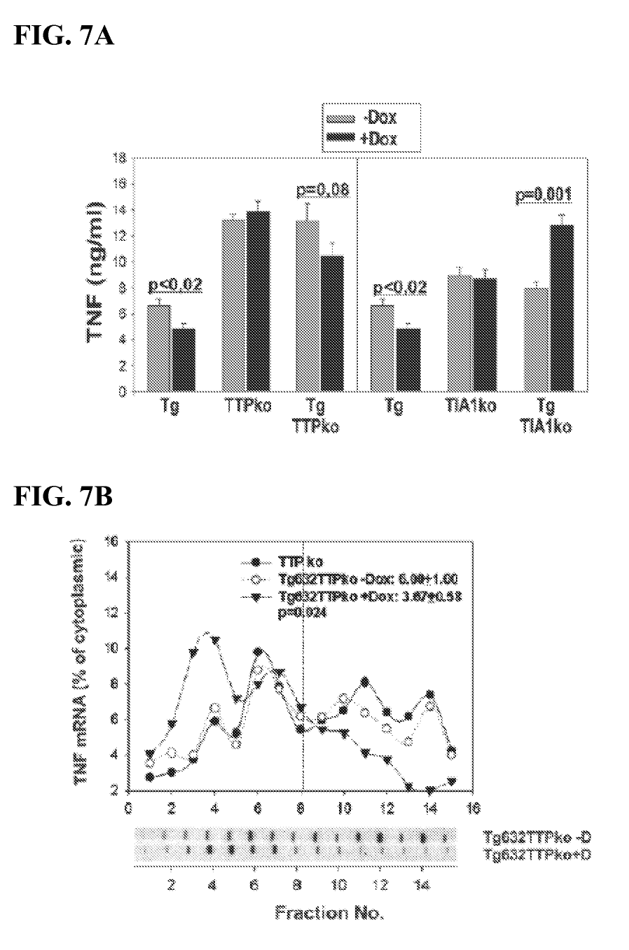

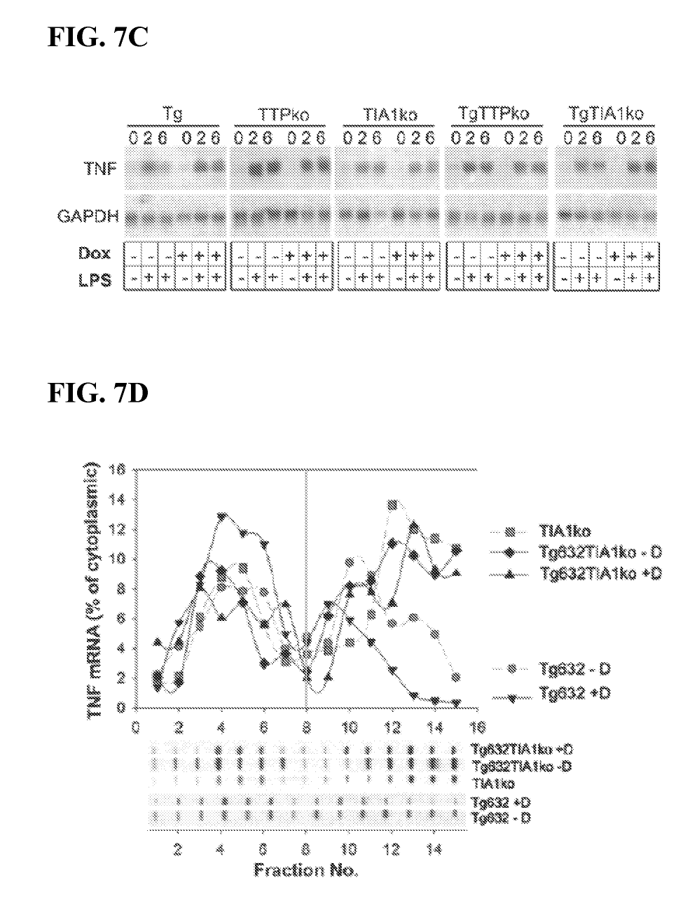

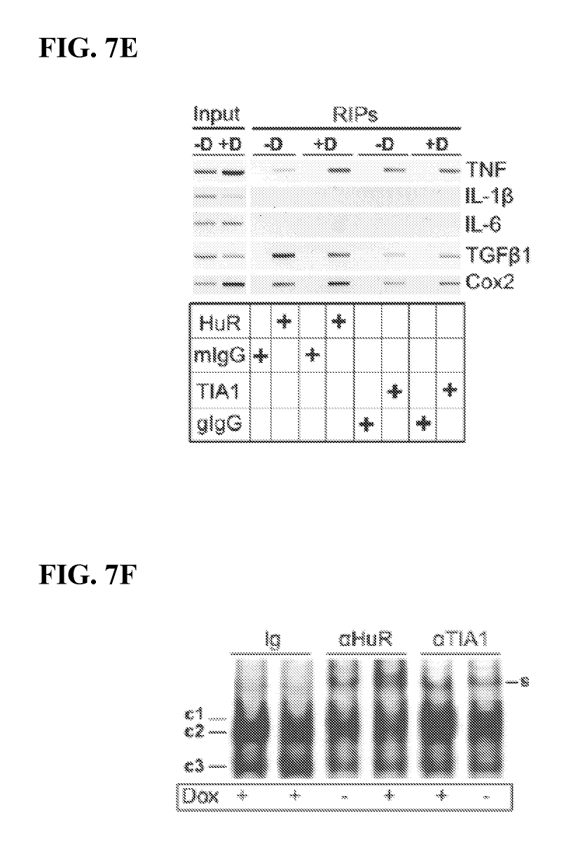

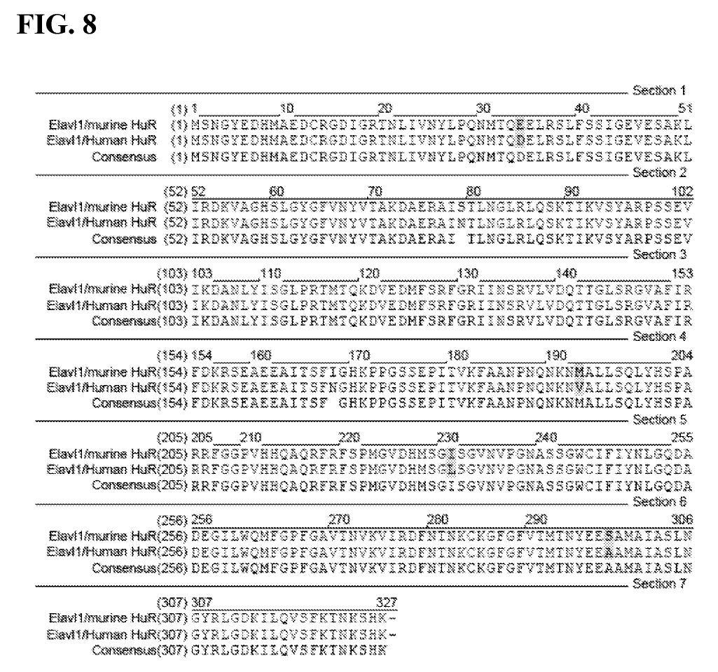

Examination of induced Tg632 BMDM protein extracts for the presence of TTP and TIA-1 indicated that HuR overexpression did not compromise their accumulation (FIG. 6G). To analyze whether HuR affects the TTP-dependant destabilization of TNF mRNA, the Tg632 HuR allele was introduced in a TTP null background. TTP^{-/-} BMDM accumulated 3 fold higher levels of TNF mRNA and produced 2 fold higher levels of TNF protein in LPS stimulated macrophages (FIG. 7A, C). The induction of HuR in Tg632⁺TTP^{-/-} BMDM, did not increase further the accumulation of TNF mRNA (FIG. 7C); however, it did result in a partial, yet significant reduction in the polysome associated TNF mRNA in these cultures (7B and data not shown). Strangely however, the secreted levels of TNF protein from Tg632⁺TTP^{-/-} BMDM were marginally, but not significantly reduced relative to the control levels from TTP^{-/-} BMDM (FIG. 7A). These results indicate that in macrophages, HuR does not affect TNF mRNA stability in the absence of TTP and suggest that the effect of HuR on translational inhibition may occur prior to the destabilizing effects of TTP.

To examine whether the translational inhibition imposed by HuR on TNF mRNA is mediated through the activities of TIA-1, the transgenic HuR allele was introduced in a TIA-1 null background. Under the LPS stimulation regime employed, TIA-1^{-/-} BMDM produced 20% more TNF protein than control macrophages, with no apparent differences in TNF mRNA accumulation. Dox stimulation did not alter TNF biosynthesis in TIA-1^{-/-} BMDM, whereas the response Tg632⁺TIA-1^{+/+} BMDMs was as previously described. The activation of transgenic HuR in Tg632⁺TIA-1^{-/-} BMDM increased the TNF mRNA pool as in Tg632⁺TIA-1^{+/+} controls (FIG. 7C); however, examination of the polysome profiles from all groups of BMDM demonstrated that the previously reported increased association of the TNF mRNA in the absence of TIA-1, was not altered in induced Tg632+TIA-1^{-/-} BMDM as it did in Tg632⁺TIA-1^{+/+} controls (FIG. 7D) and Tg632⁺TIA-1^{-/-} BMDM produced 60% more TNF protein than TIA1^{-/-} macrophages (FIG. 7A). This data suggests that HuR synergizes-genetically and functionally—with TIA-1 to reduce the translation of TNF mRNA. To gain more insight on the putative cooperative functions between HuR and TIA-1 we performed RIP assays with TIA-1 (see also FIG. 6F) to examine for its association with the HuR cognate targets identified herein. As demonstrated in FIG. 7E (& Supplemental FIG. 2) the mRNAs of TNF, TGFβ1 and Cox2 were detected in RIP assays with anti-TIA-1 antisera in transgenic and RAW264.7 macrophages, as they did in the case of RIPs with the anti-HuR antisera. In addition and as demonstrated in FIG. 6E, an association between TIA-1 and the TNF^{ΔARE} mRNA could not be observed in Tnf^{ΔARE/ΔARE} TnfRI^{-/-} BMDM, whereas Cox2 mRNA was observed in all TIA1-IPs. Finally, and as demonstrated in FIG. 7F, TIA-1 was detected in supeshift mobility assays with extracts from Tg632⁺ BMDM.

### Experimental Procedures

Transgene Preparation. For the effector transgene, a 3.5 Kb BglII/HincII human lysozyme promoter fragment (Clarke et al., 1996) was placed in front of rtTAS2 sequence (Urlinger et al., 2000). Intronic and poly-adenylation sequences were attached at the 3′ in the form of a defective human growth hormone gene (Iritani et al, 1997). For the target transgene, the complete cDNA sequence for human HuR was obtained from IMAGE clone # 2901220 (GeneBank Acc No. BCoo3376) and was subcloned in frame to an HA epitope tag for the expression of an N-terminally tagged form. Subsequently, the HA-HuR cDNA was used for the generation of a transgenic construct that contained in a 5′ to 3′ orientation (a) seven copies of the 42-bp tet operator sequence upstream of the minimal CMV promoter (Clontech); (b) a β-globin intronic sequence for optimal transgene expression; (c) the HA-HuR cDNA and (d) a bovine growth hormone polyadenylation signal. For the production of transgenic mice, fertilized CBAxC57BI/6 hybrid (F2) zygotes were co-injected with the transgenic devices using standard procedures. To identify and maintain transgenic founder mice, tail DNA was used for southern blot hybridizations and PCR using specific probes and primers for the detection of the transgenes.

Targeted mutant mice. The generation of (B6, 129Sv) Tnf^{ΔARE/-}, (B6) Tnf^{ΔARE}/^{ΔARE}TnfRI^{-/-}, (B6) TTP^{-/-} and (B6) TIA-1^{-/-} mutant mice has been previously described (Kontoyiannis et al., 1999; Taylor et al, 1996; Piecyk et al., 2000). All mice were bred and maintained in the animal facilities of the BSRC “Alexander Fleming” under specific pathogen-free conditions.

Cell isolation and Culture. Total exudate peritoneal macrophages (TEPM) and bone marrow macrophages (BMDM) were isolated as previously described (Kontoyiannis et al., 1999). Total splenocytes, lung exudate cells, peripheral blood monocytes and peritoneal cavity cells were collected from 6-8 wk old-mice and cultured in single cell suspensions as described in protocols included in Paulnock et al (2000). For all macrophage experiments, cells were seeded at a density of 5×10⁵ cells/well in 24 well tissue culture plates or 1×10⁷ cells/10 cm² plates. Doxycycline (D-9891) was purchased from Sigma. Since Dox, can inhibit macrophages responses at high concentrations (>25 μg/ml) (Attur et al., 1999), we used a 1-10 μg/ml range for 24 hrs that did not appear to affect macrophage responses in control cultures (not shown), but induced the transgenic protein in dose dependant manner. LPS (Salmonella enteriditis; Sigma) was used at a concentration of 100 ng/ml for the indicated time periods post Dox administration.

RNA analysis. Total RNA was extracted from mouse organs or cell cultures using Trizol Reagent (InVitrogen) according to manufacturer's instructions. For RT-PCR, 5-10 μg of tissue or cellular RNA were used for cDNA synthesis with MMLV-RT (Promega). cDNA products were then used for the detection of the transgenic or cytokine transcripts with specific combination of primers (Supplemental table 3). For the detection of cytokine and chemokine messages using RPAs see Supplemental Table 1. For Northern analysis, RNA samples were either analyzed through denaturing agarose electrophoresis and transfer onto nylon Hybond membranes (Amersham) or blotted directly onto these membranes using a slot blot apparatus. Blots where hybridized with PCR amplified or random primed ³²P-labelled probes for the selected mRNAs using standard techniques. Isolation of macrophage nuclei and nuclear run-on reactions were performed as previously described (Kontoyiannis et al., 1999). For polysome analysis, cytoplasmic fractions containing monosomes and polysomes were isolated as previously described (Kontoyiannis et al., 2001) from cultured macrophages. RNA was extracted from each fraction and used for analysis.

Protein Analysis. For Western analysis, whole cell lysates were prepared in Laemmli buffer whereas nuclear and cytoplasmic extracts were prepared using the NE-PER reagent (Pierce) according to manufacturer's instructions. Equimolar amounts of protein were analyzed on 12% polyacrylamide gel and blotted onto nitrocellulose membrane (Schleicher & Schuell). The membranes were sequentially probed with the following primary antibodies for: HuR (3A2), TIA-1 (C-20), U1SnRNP (C-18), Actin (C-11) and α-Tubulin (H-300) from Santa Cruz Biotech, HA (HA.11) from Covance, Cox-2 from Cayman chemicals and TTP as described in Cao et al., 2004. Primary antibodies were detected using horseradish peroxidase conjugated secondary antibodies (Southern Biotechnologies) by enhanced chemiluminescence (SuperSignal, Pierce). For the cytometric detection of proteins, cells were initially stained with CD11b (Pharmigen), fixed in 2% PFA for 30 min, permeabilized using microwaves for 15 sec, stained with antiserum for HuR or the HA epitope tag and analyzed on a Coulter EPICS-XL Flow Cytometer. mTNF levels in macrophage supernatants or mouse sera were measured using a TNF ELISA as previously described (Kontoyiannis et al., 1999). mIL-1β (Endogen), mIL-6 (Endogen), mTGFβ1 (R&D systems) levels were measured using ELISA kits according to manufacturer's instructions.

RNA:protein interactions. For mobility shift assays, cytoplasmic protein extracts where isolated from stimulated macrophage cultures using the NE-PER reagents (PIERCE) according to manufacturer's instructions. Binding reactions were performed as described by Dean et al. (2001), using 8 μg of cytoplasmic extract and 6×10⁴ cpm of ³²P-labeled RNA corresponding to the human TNF 3′ARE or a mutant control (Supplemental FIG. 1), and subsequent digestion with RNAse T1. For supershifts, 200 ngs of antibody were added to the reaction prior to RNAse T1 digestion. Complexes were separated in 5% native acrylamide:bis (29:1) gel and visualized using a STORM Phosphorimager device. For R-IP assays, mRNP lysates were prepared from macrophage populations and IPs with anti-HuR (3A2), anti-TIA1 (C-20) and anti-HA (HA.11) were performed as described in Tenenbaum et al., (2002) using 30 μg of specific or control antibody (mouse, goat and rabbit IgG respectively—Sigma). RNA was extracted from the IP material and used to perform semi-quantitative RT-PCR using specific primers.

ConA-induced hepatitis. Male age matched (2-3 mo) wild type and transgenic littermates (B6, CBA) were fed at libidum with 2 mg/ml Doxycycline or 5% sucrose diluent for 3 weeks prior of a single intravenous injection of 30 mg/Kg Concanavalin A (Sigma). Liver tissue samples and sera from individual mice were obtained at 24 h and 48 h post injection. The level of the AST in the serum was determined by a biochemical kit (Linear Chemicals) according to manufacturer's instruction.

Experimental Procedures for Sup. FIG. 1

Generation of recombinant GST-HA-HuR protein. For the production of recombinant GST-HA-HuR protein, pGEX-HuR vector was used, as described by Ma et al. (1996) by replacing HuR with the HA-HuR cDNA that was used for the transgene generation. Constructs were expressed in BL-21 bacterial strain and were purified, 3 h after IPTG induction to a final concentration of 1 mM, using the MagneGST Protein Purification System (Promega) according to manufacturer's instructions.

RNA:Protein interactions. The mobility shift assays were performed as described in the main body of Materials and Methods.

Experimental Procedures for Sup. FIG. 2

The murine myelomonocytic cell line RAW264.7 was acquired from ATCC (USA) and maintained in RPMI1640+5% FBS medium. Prior to experimentation, cells were seeded at a density 1-5×10⁷ cells/10 cm² tissue culture plate. mRNP isolation and RIP assays were performed as described in the main text.

Histology. Paraffin embedded liver tissue samples were sectioned and stained with hematoxylin and eosin.

Statistics. The unpaired student's t-test for statistical significance was used to compare protein and mRNA values.

SUPPLEMENTARY TABLE 1
Analysis of mRNA populations affected by the induction of
transgenic HuR in LPS stimulated mouse macrophages.

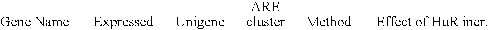

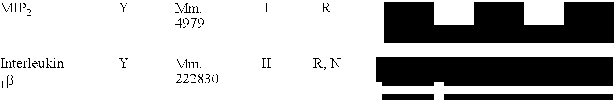

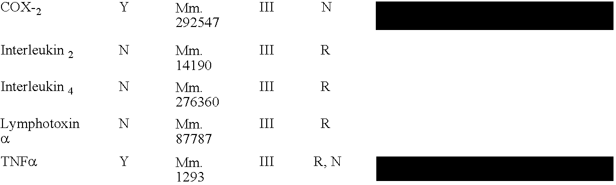

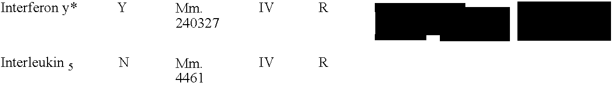

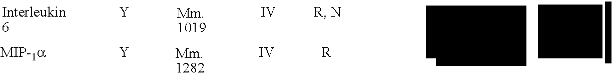

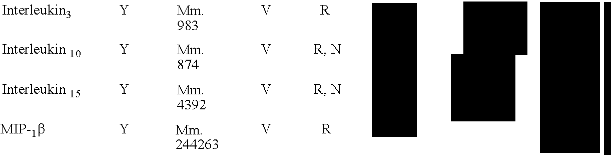

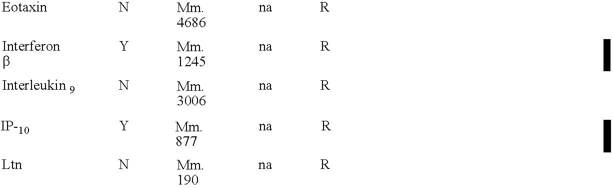

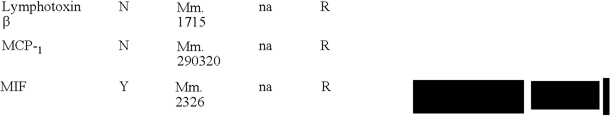

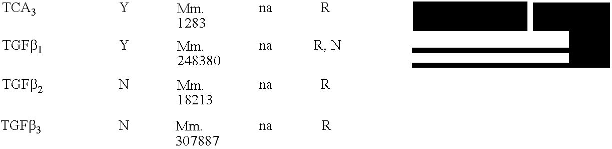

Cytokine and chemokine transcripts examined in 10 μg of total RNA isolated from LPS stimulated transgenic macrophage cultures using Ribonuclease Protection Assays (R) and Northern Blots (N). Indicated are the messages detected (italics), their corresponding Unigene annotation numbers, the cluster of AU-rich elements that they contain according to the ARED database and the type of variation that was observed following the induction of transgenic HuR. White-not detected; Gray-no variation; Green-downregulated; Red-upregulated. *IFNy mRNA was detected in traceable amounts. Methods: 5 μg of cellular RNA were used for Ribonuclease protection analysis using the Riboquant probes (Pharmigen) mCK₁b, mCK-₃b. mCK-₅c and the RPAIII kit (Ambion) according to manufacturer's instructions. Protected ³²P-labeled fragments were analyzed in 5% acrylamide-urea gels and visualized using a STORM Phosphorimager device. Northern blots were performed as described in the main text.

SUPPLEMENTARY TABLE 2
Measurements of LPS-induced Lethality

| | |
|---|---|
| Dose | Lethality (deaths/total) |

| | Dox | | Tg6₃2 |
|---|---|---|---|
| LPS | mg/ml | B6, CBA | B6, CBA |
| 400 | — | 0/5 | 0/5 |
| 400 | 2 | 0/5 | 0/5 |
| 800 | — | 0/5 | 0/5 |
| 800 | 2 | 0/5 | 0/5 |
| 1000 | — | 0/10 | 0/10 |
| 1000 | 2 | 0/5 | 0/5 |
| 1200 | — | 8/15 | 9/15 |
| 1200 | 2 | 3/5 | 3/5 |

Transgenic and control littermate mice (10-12 wk of age) were fed ad libidum with 2 mg/ml Dox or with sucrose diluent for 3 weeks and then were injected intraperitoneally with the indicated amounts of LPS (Salmonella enteritidis, L-6011; Sigma). The dose of LPS was calculated per 20 g of body weight. Survival was monitored for a period of 7 days. Fisher's Exact test did not indicate any statistical difference amongst any of the groups.

SUPPLEMENTARY TABLE 3
List of primers used in this study

| Name | Sequence |
|---|---|
| rtTA sense | ₅′GCAAAAGAGGAAAGAGAGAC₃′ |
| rtTA antisense | ₅′ATAAGGGAATGGTTGGGAAG₃′ |
| HA-hHuR sense | ₅′TTTACCCCTACGACGTCCCC₃′ |
| HA-hHuR antisense | ₅′TCCTTCGCGGTCACGTAGTT₃′ |
| mHuR/Elavl₁ sense | ₅′ATGTCTAATGGTTATGAAGA₃′ |
| mHuR/Elavl₁ antisense | ₅′AGCTTTGCAGATTCAACCTC₃′ |
| mGAPDH sense | ₅′TCTTCTTGTGCAGTGCC₃′ |
| mGAPDH antisense | ₅′ACTCCACGACATACTCAGC₃′ |
| mTNF sense | ₅′CACGCCTCTTCTGTCTACTGAACTTCG₃′ |
| mTNF antisense | ₅′GGCTGGGTAGAGAATGGATGAACACC₃′ |
| mCOX₂ sense | ₅′ATGCTCTTCCGAGCTGTGCT₃′ |
| mCOX₂ antisense | ₅′GCCCACATCTATGCTTTTCA₃′ |
| mTTP sense | ₅′CAGAGCCTCCAGTCGATGAG₃′ |
| mTTP antisense | ₅′GAAGATGGGGAGACGCCTTG₃′ |
| mIL-6 sense | ₅′TGGAGTCACAGAAGGAGTGGCTAAG₃′ |
| mIL-6 antisense | ₅′TCTGACCACAGTGAGGAATGTCCAC₃′ |
| mIL-_{1β}sense | ₅′CTGAAGCAGCTATGGCAACT₃′ |
| mIL-_{1β}antisense | ₅′GGATGCTCTCATCTGGACAG₃′ |
| mTGF_{β1}, sense | ₅′TTCCTGCTCCTCATGGCC₃′ |
| mTGF_{β1}, antisense | ₅′CTTGCAGGAGCGCACGAT₃′ |
| β-actin | DNA fragment (Ambion Cat. #7323G1) |

Supplementary Data Set: UTRs with HBMs & AREs

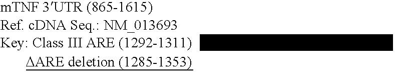

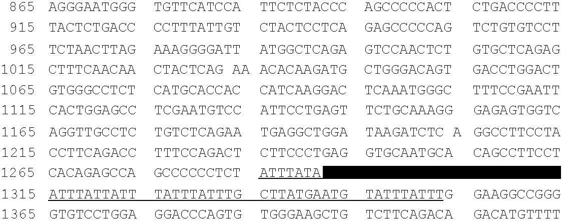

1615ATAAA

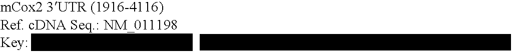

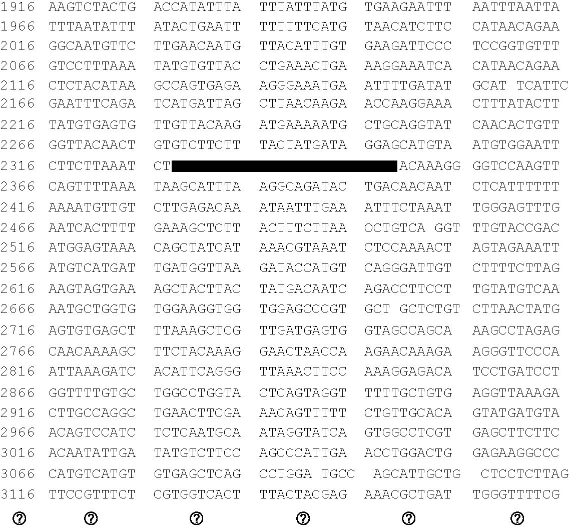

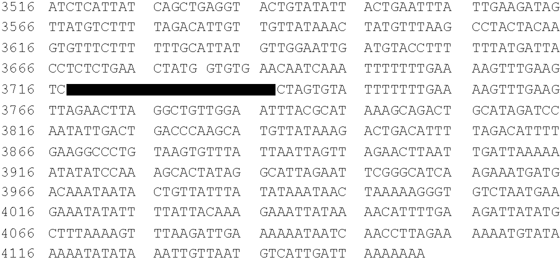

### REFERENCES

Anderson, P. and Kedersha, N. (2002). Visibly stressed: the role of eIF2, TIA-1, and stress granules in protein translation. Cell Stress. Chaperones. 7, 213-221.
Attur, M. G., Patel, R. N., Patel, P. D., Abramson, S. B., and Amin, A. R. (1999). Tetracycline up-regulates Cox-2 expression and prostaglandin E2 production independent of its effect on nitric oxide. J. Immunol. 162, 3160-3167.
Bonizzi, G. and Karin, M. (2004). The two NF-kappaB activation pathways and their role in innate and adaptive immunity. Trends Immunol. 25, 280-288.
Brennan, C. M. and Steitz, J. A. (2001). HuR and mRNA stability. Cell Mol. Life Sci. 58, 266-277.
Brooks, S. A., Connolly, J. E., and Rigby, W. F. (2004). The role of mRNA turnover in the regulation of tristetraprolin expression: evidence for an extracellular signal-regulated kinase-specific, AU-rich element-dependent, autoregulatory pathway. J. Immunol. 172, 7263-7271.
Cao, H., Tuttle, J. S., and Blackshear, P. J. (2004). Immunological characterization of Tristetraproline as a low abundance, inducible, stable cytosolic protein. J. Biol. Chem. 279, 21489-21499.
Carballo, E., Lai, W. S., and Blacksbear, P. J. (1998). Feedback inhibition of macrophage tumor necrosis factor-alpha production by tristetraprolin. Science 281, 1001-1005.
Carballo, E., Lai, W. S., and Blackshear, P. J. (2000). Evidence that tristetraprolin is a
Clarke, S., Greaves, D. R., Chung, L. P., Tree, P., and Gordon, S. (1996). The human lysozyme promoter directs reporter gene expression to activated myelomonocytic cells in transgenic mice. Proc. Natl. Acad. Sci. U.S.A % 20; 93, 1434-1438.
Cok, S. J., Acton, S. J., Sexton, A. E., and Morrison, A. R. (2004). Identification of RNA-binding proteins in RAW 264.7 cells that recognize a lipopolysaccharide-responsive element in the 3-untranslated region of the cyclooxygenase-2 mRNA. J. Biol. Chem. 279, 8196-8205.
De Silanes, L, Zhan, M., Lal, A., Yang, X. and Gorospe, M. (2004). Identification of a target RNA motif for RNA-binding protein HuR. Proc. Natl. Acad. Sci. U.S.A 101, 2987-2992.
Dean, J. L., Wait, R., Mahtani, K. R., Sully, G., Clark, A. R, and Saklatvala, J. (2001). The 3′ untranslated region of tumor necrosis factor alpha mRNA is a target of the mRNA-stabilizing factor HuR. Mol. Cell Biol. 21, 721-730.
Di Marco S, Hel Z, Lachance C, Furneaux H and Radzioch D. (2001). Polymorphism in the 3′-untranslated region of TNFalpha mRNA impairs binding of the post-transcriptional regulatory protein HuR to TNFalpha mRNA. Nucleic Acids Res. 29, 863-871.
Dixon, D. A., Balch, G. C., Kederslia, N., Anderson, P., Zimmerman, G. A., Beauchamp, R. D., and Prescott, S. M. (2003). Regulation of cyclooxygenase-2 expression by the translational silencer TIA-1. J. Exp. Med. 198, 475-481.
Fan, X. C. and Steitz, J. A. (1998). HNS, a nuclear-cytoplasmic shuttling sequence in HuR. Proc. Natl. Acad. Sci. U.S.A 95, 15293-15298.
Gallouzi, I. E., Brennan, C. M., Stenberg, M. G., Swanson, M. S., Eversole, A., Maizels, N., and Steitz, J. A. (2000). HuR binding to cytoplasmic mRNA is perturbed by heat shock. Proc. Natl. Acad. Sci. U.S.A 97, 3073-3078.
Glauser, M. P. (1996). The inflammatory cytokines. New developments in the pathophysiology and treatment of septic shock. Drugs 52 Suppl 2:9-17, 9-17.
Iritani, B. M., Forbush, K. A., Farrar, M. A., and Perlmutter, R. M. (1997). Control of B cell development by Ras-mediated activation of Raf. EMBO J. 16, 7019-7031.
Keene, J. D. and Tenenbaum, S. A. (2002). Eukaryotic mRNPs may represent posttranscriptional operons. Mol. Cell 9, 1161-1167.
Kontoyiannis, D., Pasparakis, M., Pizarro, T. T., Cominelli, F., and Kollias, G. (1999). Impaired on/off regulation of TNF biosynthesis in mice lacking TNF AU-rich elements: implications for joint and gut-associated immunopathologies. Immunity. 10, 387-398.
Kontoyiannis, D., Kotlyarov, A., Carballo, E., Alcxopoulou, L., Blackshear, P. J., Gaestel, M., Davis, R., Flavell, R., and Kollias, G. (2001). Interleukin-10 targets p38 MAPK to modulate ARE-dependent TNF mRNA translation and limit intestinal pathology. EMBO J. 20, 3760-3770.
Kullmann, M., Gopfert, U., Siewe, B., and Hengst, L. (2002). ELAV/Hu proteins inhibit p27 translation via an IRES element in the p27 S′UTR. Genes Dev. 16, 3087-3099.
Lal, A., Mazan-Mamczarz, K., Kawai, T., Yang, X., Martindale, J. L., and Gorospe, M. (2004). Concurrent versus individual binding of HuR and AUF1 to common labile
Mazan-Mamczarz, K, Galban, S., Lopez, d. S., I, Martindale, J. L., Atasoy, U., Keene, J. D., and Gorospe, M. (2003). RNA-binding protein HuR enhances P53 translation in response to ultraviolet light irradiation. Proc. Natl. Acad. Sci. U.S.A 100, 8354-8359.
McMullen, M. R., Cocuzzi, E., Hatzoglou, M., and Nagy, L. E. (2003). Chronic ethanol exposure increases the binding of HuR to the TNFalpha 3′-untranslated region in macrophages. J. Biol. Chem. 278, 38333-38341.
Paulnock, D. M. (2000). Macrophages: A Practical Approach (New York: Oxford University Press)
Piecyk, M., Wax, S., Beck, A. R., Kedersha, N., Gupta, M., Maritim, B., Chen, S., Gueydan, C., Kruys, V., Streuli, M., and Anderson, P. (2000). TIA-1 is a translational silencer that selectively regulates the expression of TNF-alpha. EMBO J. 19, 4154-4163.
Saklatvala, J. (2004). The p38 MAP kinase pathway as a therapeutic target in inflammatory disease. Curr Opin Pharmacol. 4, 372-377
Sass, G., Heinlein, S., Agli, A., Bang, R., Schumann, J., and Tiegs, G. (2002). Cytokine expression in three mouse models of experimental hepatitis. Cytokine 19, 115-120.
Stoecklin, G., Stubbs, T., Kedersha, N., Wax, S., Rigby, W. F., Blackwell, T. K., and Anderson, P. (2004). MK2-induced tristetraprolin: 14-3-3 complexes prevent stress granule association and ARE-mRNA decay. EMBO J. 23, 1313-1324.
Sun, R., Tian, Z., Kulkarni, S., and Gao, B. (2004). IL-6 prevents T cell-mediated hepatitis via inhibition of NKT cells in CD4+ T cell- and STAT3-dependent and autoimmunity resulting from tristetraprolin (TTP) deficiency. Immunity. 4, 445-454.
Tenenbaum, S. A., Lager, P. J., Carson, C. C., and Keene, J. D. (2002). Ribonomics: identifying mRNA subsets in mRNP complexes using antibodies to RNA-binding proteins and genomic arrays. Methods 26, 191-198.
Urlinger, S., Baron, U., Thellmann, M., Hasan, M. T., Bujard, H., and Hillen, W. (2000). Exploring the sequence space for tetracycline-dependent transcriptional activators: novel mutations yield expanded range and sensitivity. Proc. Natl. Acad. Sci. U.S.A 97, 7963-7968.
Wilusz, C. J., Wormington, M., and Peltz, S. W. (2001). The cap-to-tail guide to mRNA turnover. Nat. Rev. Mol. Cell Biol. 2, 237-246.
Winzen, R, Gowrishankar, G., Bollig, F., Redich, N., Resch, K., and Holtmann, H. (2004). Distinct domains of AU-rich elements exert different functions in mRNA destabilization and stabilization by p38 mitogen-activated protein kinase or HuR Mol. Cell Biol. 24, 4835-4847.
W J Ma, S. Cheng, C. Campbell, A. Wright, H. Furneaux (1996) Cloning and characterization of HuR, a ubiquitously expressed Elav-like protein J. Biol Chem. 5; 271(14): 8144-51.

1. A method for modulating translation of a specific mRNA in a cell comprising modulating expression of a member of the Elavl1/Hu family of selective RNA binding proteins (RBPs) in said cell and incubating said cell to allow transcription of said specific mRNA.

2. A method according to claim 1 wherein said translation of a specific mRNA in a cell is reduced by over-expressing a member of the Elavl1/Hu family of selective RNA binding proteins (RBPs) in said cell.

3. A method according to claim 1 wherein said specific mRNA comprises a binding site for said member.

4. A method according to claim 1, wherein said mRNA encodes a cytokine.

5. A method according to claim 1, wherein said member is a human primate member of the Elavl1/Hu family.

6. A method according to claim 1, wherein said cell is a human primate cell.

7. A method according to claim 1, wherein said cell is an immune cell.

8. A method according to claim 7, wherein said cell is a cell of the myeloid lineage.

9. A method according to claim 1, wherein said cell produces a cytokine in at least a part of its life.

10. A method according to claim 1, wherein said cell is provided with a nucleic acid comprising a coding region for said member.

11. A method according to claim 1, wherein said member comprises HuR or a functional part or derivative thereof.

12. A method according to claim 1, wherein said cell is a cell of a transgenic non-human mammal wherein said non-human mammal comprises a heterologous nucleic acid sequence encoding said member of the Elavl1/Hu family.

13. A method for stabilizing specific mRNA in a cell comprising over-expressing a member of the Elavl1/Hu family of selective RNA binding proteins (RBPs) in said cell and incubating said cell to allow transcription of said specific mRNA.

14. A method according to claim 13, wherein said specific mRNA comprises a Hu-R binding motif and a type III ARE sequence.

15. A method for modifying a rate of translation of specific mRNA in a cell comprising altering an amount of a member of the Elavl1/Hu family in said cell.

16. A method according to claim 19 wherein said method dampens an inflammatory response in said individual.

17. A method according to claim 19 wherein said method dampens an auto-immune inflammatory response in said individual.

18. A method according to claim 19 wherein said individual suffers from rheumatoid arthritis.

19. A method for the treatment of an individual suffering from inflammation symptoms in at least a part of the body, said method comprising providing said individual with a gene delivery vehicle comprising nucleic acid encoding a member of the Elavl1/Hu family.

20. A method according to claim 19, wherein said gene delivery vehicle is administered locally at a site of inflammation.
